(19) United States
(12) Patent Application Publication
(10) Pub. No.: US 2019/0053761 A1
(43) Pub. Date: Feb. 21, 2019

(54) SYSTEMS AND METHODS FOR MONITORING A SUBJECT AT REST

(71) Applicant: Select Comfort Retail Corporation, Minneapolis, MN (US)

(72) Inventors: Steven Jay Young, Los Gatos, CA (US); Marco Kenneth Della Torre, San Francisco, CA (US); Yuri Zhovnirovsky, Campbell, CA (US); Richard Vincent Rifredi, Los Gatos, CA (US)

(21) Appl. No.: 16/040,724

(22) Filed: Jul. 20, 2018

Related U.S. Application Data
(63) Continuation of application No. 15/391,117, filed on Dec. 27, 2016.
Application No. 15/391,117 is a continuation of application No. 13/035,397, filed on Feb. 25, 2011.
Application No. 13/035,397 is a continuation-in-part of application No. 11/849,051, filed on Aug. 31, 2007, now abandoned.
Application No. 13/035,397 is a continuation-in-part of application No. 12/349,167, filed on Jan. 6, 2009, now abandoned.
(60) Provisional application No. 61/406,262, filed on Oct. 25, 2010.
(60) Provisional application No. 60/846,642, filed on Sep. 22, 2006.

(51) Int. Cl. A61B 5/00; A61B 5/11; A61B 5/0205; A61B 5/024; A61B 5/08

(52) U.S. Cl. CPC A61B 5/6892; A61B 2562/0247; A61B 5/7435; A61B 5/11; A61B 5/1102; A61B 5/0205; A61B 5/4818; A61B 5/4815; A61B 5/447; A61B 5/1117; A61B 5/6887; A61B 5/024; A61B 5/0816; A61B 5/1115; A61B 2562/168; A61B 5/743

(57) ABSTRACT

Disclosed herein are methods and devices for monitoring a subject at rest. One such device comprises a sensing unit having a fluid-filled bladder configured to be placed under a substrate on which the subject lays and a sensor in fluid communication with the bladder. The sensor is configured to sense pressure variations within the bladder generated by a heart beat, respiration and body weight of the subject and to generate signals indicative of the pressure variations. A processor is configured to receive the signals and to determine and generate output indicative of the subject's heart beat and respiration and presence on the substrate. An external device is configured to display one or more of the output.

### CROSS REFERENCE TO RELATED APPLICATIONS

This application is a Continuation of U.S. patent application Ser. No. 15/391,117 filed Dec. 27, 2016, which is a Continuation of U.S. patent application Ser. No. 13/035,397 filed on Feb. 25, 2011 which_claims priority to U.S. Provisional Patent Application Ser. No. 61/406,262 filed on Oct. 25, 2010. Application Ser. No. 13/035,397 is a Continuation-In-Part of U.S. patent application Ser. No. 11/849,051 filed on Aug. 31, 2007, which claims priority to U.S. Provisional Application Ser. No. 60/846,642 filed on Sep. 22, 2006, and is a continuation-in-part of U.S. patent application Ser. No. 12/349,167 filed on Jan. 6, 2009, all of which are incorporated herein by reference.

### FIELD OF THE INVENTION

The present invention pertains to monitoring systems and programs for monitoring a subject or subjects at rest, and methods of performing the same.

### BACKGROUND

Historically, monitoring vital signs of a person has required expensive equipment, such as an electrocardiogram (EKG) or a ballistocardiograph (BCG). In addition to being prohibitively expensive for many situations (e.g., home use), both EKGs and BCGs can be too cumbersome for use outside of medical facilities. EKGs, for example, typically necessitate attaching electrodes to the bodies of users, while BCGs rely on large, heavy, and unaesthetic force-measuring platforms that users lie on.

In more recent times, devices including piezoelectric films or arrays of sensors have been developed to measure heart and respiration rates. A user can lie on the device, and the film or sensors can generate a signal indicate of the user's heart rate and/or respiration rate. However, these devices can also be expensive.

### SUMMARY

Disclosed herein are devices for monitoring a subject at rest. One such device comprises a sensing unit having a fluid-filled bladder configured to be placed under a substrate on which the subject lays and a sensor in fluid communication with the bladder. The sensor is configured to sense pressure variations within the bladder generated by a heart beat, respiration and body weight of the subject and to generate signals indicative of the pressure variations. A processor is configured to receive the signals and to determine and generate output indicative of the subject's heart beat and respiration and presence on the substrate. An external device is configured to display one or more of the output and may communicate with a wireless device to display output indicative of the subject's heart rate, respiration rate and presence on the substrate.

Another embodiment of the device for monitoring a subject at rest can be used to monitor patients. This embodiment comprises a sensing unit having a fluid-filled bladder configured to be placed on a substrate and a sensor in fluid communication with the bladder for generating signals in response to pressure variations within the bladder. A processing unit can be configured to receive the signals from the sensor and to generate from the signals a heart rate, respiration rate and static pressure of the subject. Means for providing to a user analyses based on the subject's heart rate, respiration rate and static pressure is included, with the analyses being an indication of the subject in bed or out of bed. Means for providing an alert message to a caregiver can also be provided.

Another embodiment of a device disclosed herein can be used for preventing pressure ulcers in subjects. This embodiment can comprise a sensing unit having a fluid-filled bladder configured to be placed on a substrate and a sensor in fluid communication with the bladder for generating signals in response to pressure variations within the bladder, wherein the pressure variations are generated from a heart rate, respiration rate and body weight of the subject. A processing unit can be configured to receive the signals from the sensor and to determine from the signals a baseline heart rate, respiration rate and static pressure and to determine changes in strength of each of the heart rate, respiration rate and static pressure. Means for providing to a user analyses based on the changes is included, wherein the analyses includes an indication of a need for a user action to prevent a pressure ulcer on the subject.

Another embodiment of a device disclosed herein is a portable biosensing device for use by a subject at home comprising a bladder configured to be filled with a fluid and placed on a substrate, a sensor in fluid communication with the bladder and configured to generate signals in response to pressure variations within the bladder, wherein the pressure variations are generated from a heart rate, respiration rate and body weight of the subject resting on the fluid-filled bladder, a means for filling the bladder with fluid, a processor configured to receive the signals from the sensor and determine the subject's heart rate and respiration rate and a database for storing the heart rate and respiration rate data.

Also disclosed herein are methods of monitoring a subject at rest. One embodiment of a method disclosed herein monitors a subject by first sensing with a sensor incident pressure waves generated by the subject resting on a monitoring device, wherein the monitoring device comprises a fluid-filled bladder configured to be placed on a substrate with the sensor in fluid communication with the bladder. Signals are generated indicative of the incident pressure waves and sending the signals to a processor. A heart beat, respiration and static pressure of the subject are discriminated from analysis of the signals and an individual fingerprint is determined based on heart beat, respiration and static pressure for the subject. Whether the subject is in an assigned bed, not in the assigned bed, or in a different bed can be determined based on the presence or absence of the individual fingerprint and this can be communicated an external device.

Another method disclosed herein is an embodiment that can prevent pressure ulcers. This method comprises sensing with a sensor incident pressure waves generated by the subject resting on a monitoring device, wherein the monitoring device comprises a fluid-filled bladder configured to be placed on a substrate with the sensor in fluid communication with the bladder. Signals are generated indicative of the incident pressure waves and sending the signals to a processor and a baseline heart rate, respiration and static pressure of the subject is determined from the signals. An amount of time the subject is resting on the monitoring device is determined, and data indicating amount of movement and degree of movement of the subject while the subject is resting based on changes in static pressure of the bladder, changes in strength of heart beat and changes in strength of respiration are determined. If the subject requires an action by a user to prevent a pressure ulcer based on the data is determined and the determination is communicated to the user.

Other embodiments of methods of monitoring one or more subjects at rest are disclosed. Another method of monitoring at least one subject at rest, wherein each subject is associated with a touchless monitoring system assembly, comprises receiving at predetermined intervals data about the subject from the associated touchless monitoring system, generating content representing the subject and the subject's data and displaying the content on a display. The touchless monitoring system assembly can comprise a fluid bladder, a sensor in fluid communication with the fluid bladder, and a processor. The sensor can be configured to determine a pressure within the fluid bladder and to send a signal indicative of the pressure to the processor, which can be configured to convert the signal to data. Receiving data from the touchless monitoring system can comprise receiving at predetermined intervals the data from the processor. The data can be communicated to the user through a bedside device with visual or audio indicator or communicated to a caregiver through a wired or wireless communication channel to a mobile device or personal computer.

Also disclosed herein are embodiments of monitoring programs for monitoring at least one subject at rest on a touchless monitoring system assembly. One exemplary embodiment comprises a monitoring system program configured to receive data from the touchless monitoring system assembly and to generate content based on the data, a display configured to display the content and a user interface. The touchless monitoring system assembly can comprise a fluid bladder, a sensor in fluid communication with the fluid bladder and a processor. The sensor can be configured to determine a pressure within the fluid bladder and to send a signal indicative of the pressure to the processor, which is configured to convert the signal to data that is received by the monitoring system program.

As used herein, data can include, but is not limited to, heart rate, respiration rate, length of sleep, quality of sleep, position, presence or absence in bed, blood pressure, tossing and turning movements, rolling movements, limb movements, and weight.

### BRIEF DESCRIPTION OF THE DRAWINGS

The description herein makes reference to the accompanying drawings, wherein like reference numerals refer to like parts throughout the several views, and wherein:

FIG. 1 is a schematic view of a touchless monitoring system apparatus for monitoring a subject at rest;

FIG. 2 is a diagram of the touchless monitoring system of FIG. 1;

FIG. 3 is a method of monitoring a subject at rest;

FIG. 4 is another method of monitoring a subject at rest;

FIG. 5 is a schematic view of a portable touchless monitoring system apparatus for monitoring a subject at rest;

FIG. 6 is a schematic view of another embodiment of a touchless monitoring system apparatus for monitoring a subject at rest;

FIG. 7 is a diagram of the touchless monitoring system of FIG. 1 with a monitoring program;

FIG. 8 is a login page generated by a monitoring system program disclosed herein;

FIG. 9 is a home page display screen generated by a monitoring system program disclosed herein;

FIG. 10 is a dashboard display screen generated by a monitoring system program disclosed herein;

FIG. 11 is a live view display screen generated by a monitoring system program disclosed herein;

FIG. 12 is a trend display screen generated by a monitoring system program disclosed herein;

FIG. 13 is a settings display screen generated by a monitoring system program disclosed herein;

FIG. 14 is another embodiment of a home page display screen generated by a monitoring system program disclosed herein; and

FIG. 15 is another embodiment of a home page display screen generated by a monitoring system program disclosed herein.

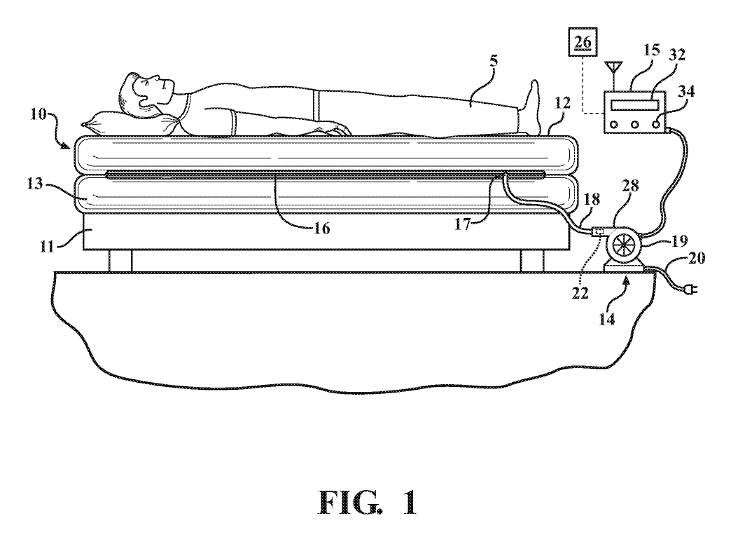

FIG. 1X is an end view of a sleep monitoring system including an air mattress and a pump;

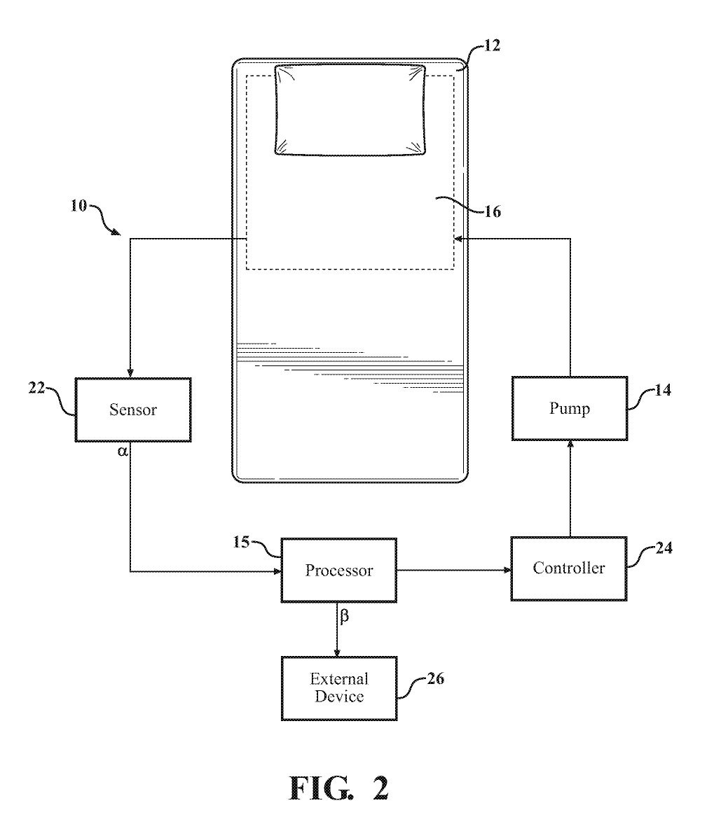

FIG. 2X is a schematic view of the sleep monitoring system of FIG. 1X;

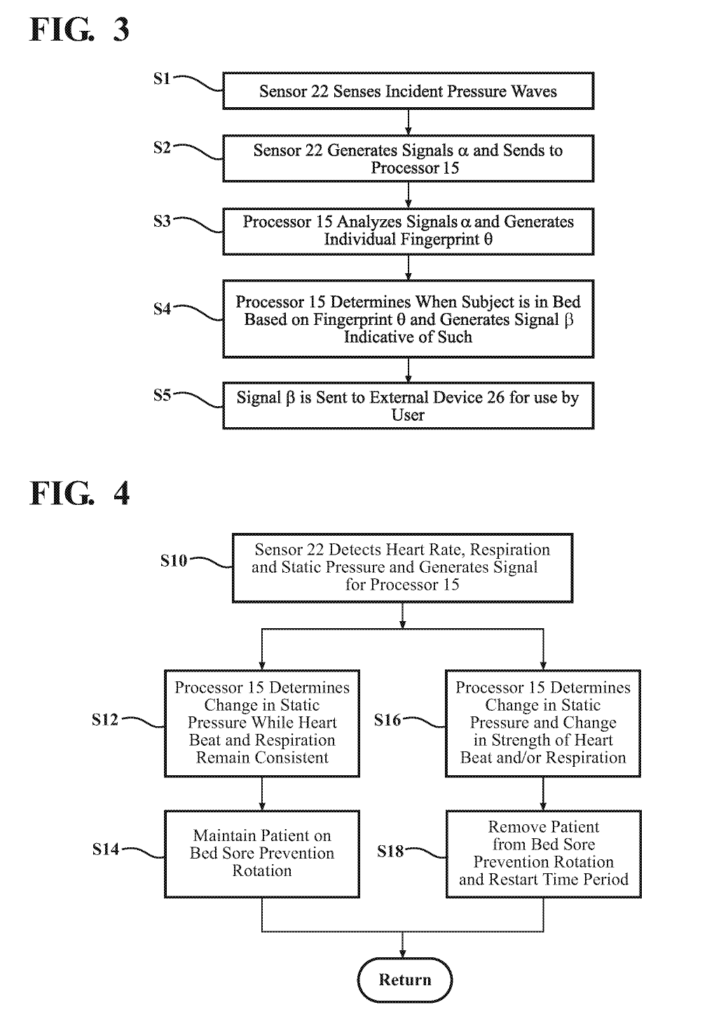

FIG. 3X is a cross-section view of the air mattress of FIG. 1X along line A-A in FIG. 1X; and

FIG. 4X is a flowchart showing a determination of a pressure setting.

FIG. 1Y is a partially schematic side or perspective view of a monitoring method and apparatus.

FIG. 2Y is a diagram illustrating how an embodiment of the system works. A pad sensing unit detects heart and respiration from the infant subject. Optional temperature and audio sensors provide additional data. A digital signal processor (DSP) analyzes data from the mattress pad sensing unit and other data.

FIG. 3Y is a diagram illustrating an embodiment of the system that utilized an air-filled mattress sensor connected to an air pump (i.e., a pressure-control unit) for maintaining pad pressure within a predefined range. Vibrations corresponding to heart and respiration functions are detected by a pressure sensor, communicated to a computer for analysis, and distributed via the internet.

FIG. 4YA-4YD shows exemplary configurations of air or fluid-filled pad sensors having a single chamber (A) or multiple chambers (B-D).

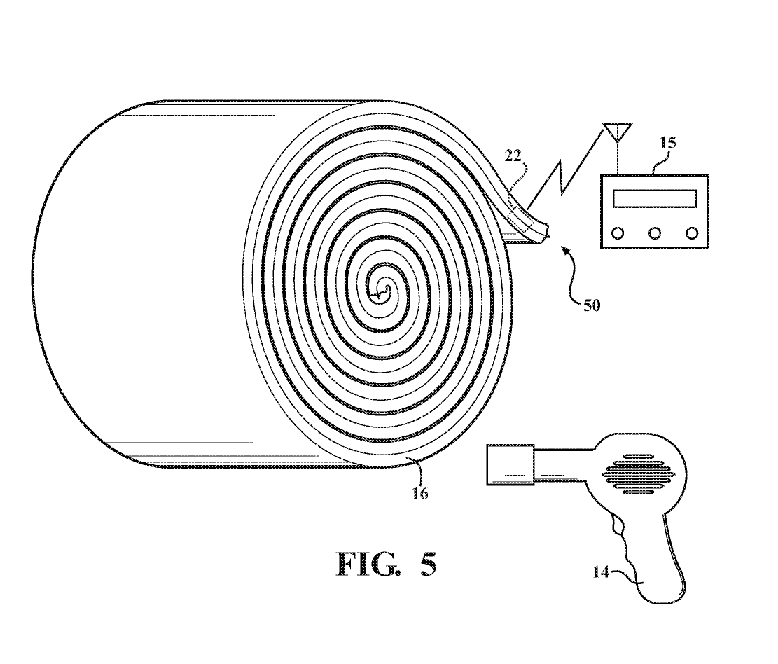

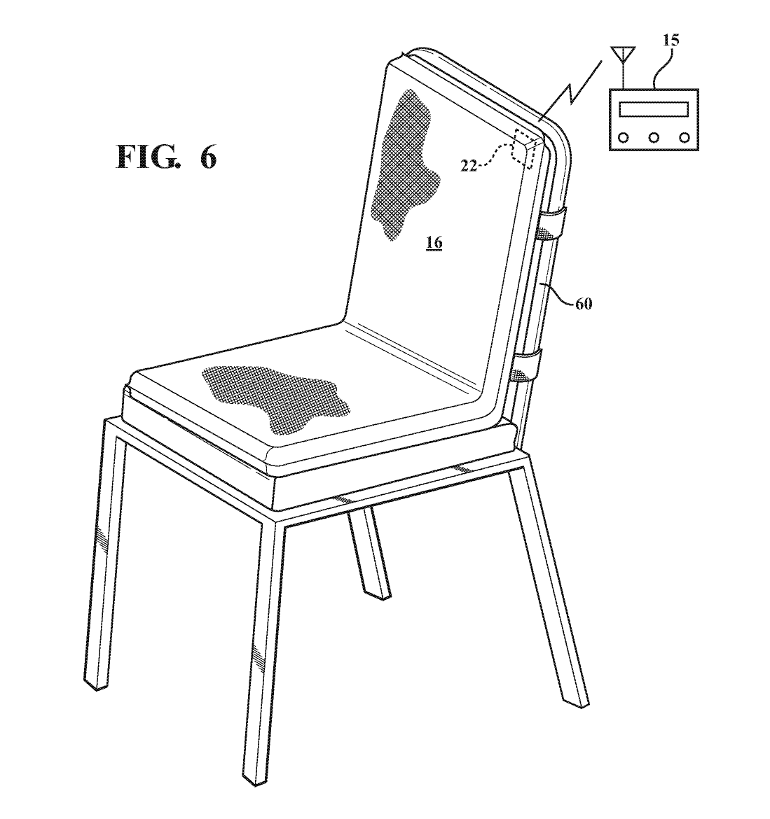

FIG. 5Y is a diagram showing how an air or fluid-filled mattress sensor is used to generate health status data. Vibrations are detected by pressure sensors, and the data are filtered and compared by a microprocessor. An ambient vibration cancellation device is also depicted.

FIG. 6Y is a schematic showing how data generated by multiple sensors are analyzed by a DSP and used to trigger events.

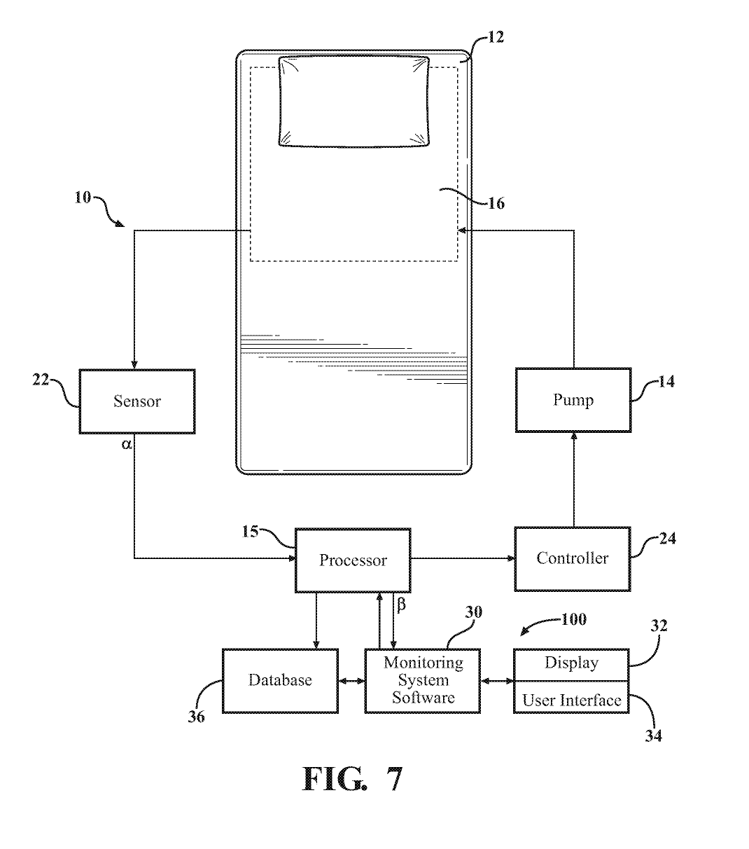

FIG. 7Y shows an example of processed data generated from a pad sensor using a 6-month-old infant subject.

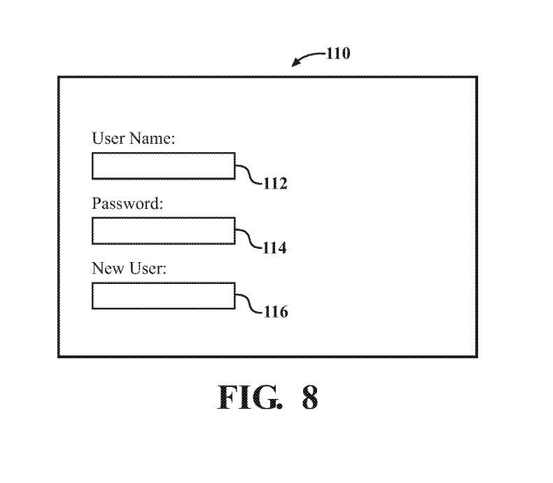

FIGS. 8YA and 8YB illustrate components of a two-plate mechanical sensor having orthogonally disposed strain gauges for monitoring heart and respiratory functions. 8YA is a side view showing an infant subject. 8YB is a top view showing the strain gauges connecting the two plates.

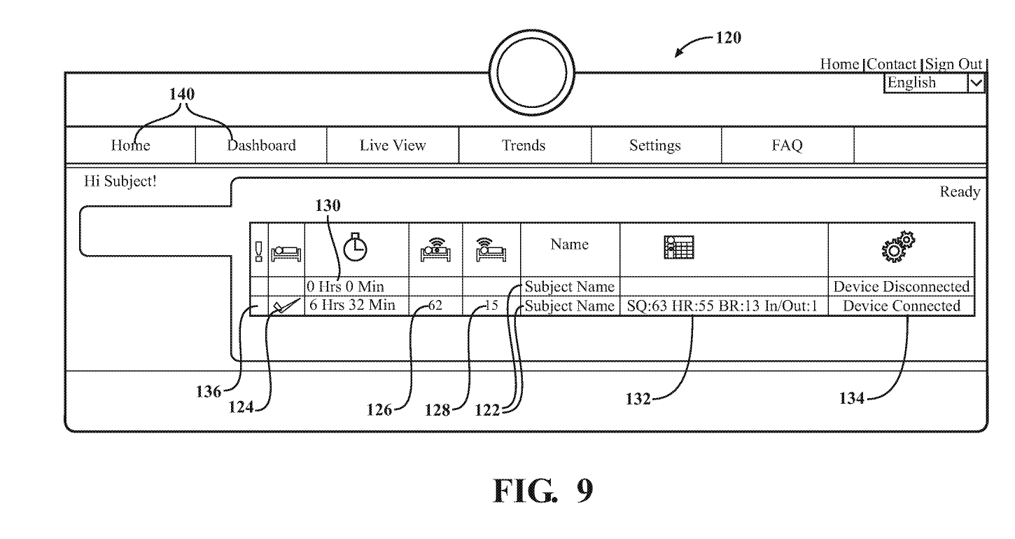

FIG. 9Y is a schematic showing how data generated by a mechanical sensor are analyzed by a DSP and used to trigger events.

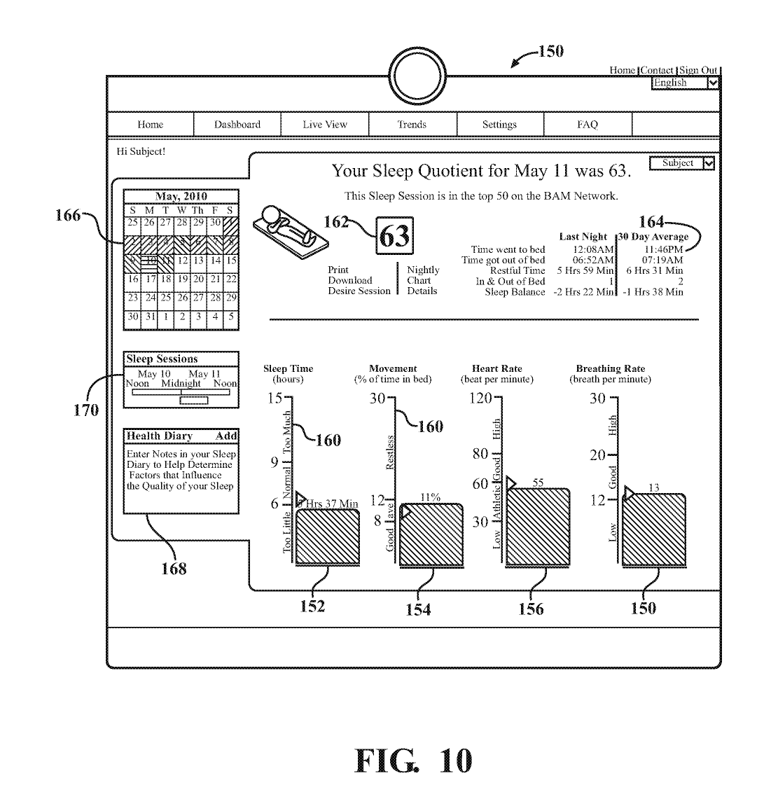

FIG. 10Y is a schematic showing how a wireless transceiver receiving data from a mattress sensor communicates with a remote microcontroller for monitoring and responding to health status data.

### DETAILED DESCRIPTION

Disclosed herein are embodiments of a monitoring system for monitoring at least one parameter of a resting subject. The monitoring system is a touchless system, meaning it does not come in contact with the subject. Conventional monitoring systems have electrodes, pressure cuffs, and the like that are placed on the subject. The touchless system can be transported for use by the same subject in different locations. The monitoring system includes the touchless monitoring apparatus and a monitoring program that receives, saves, manipulates and displays various data as content for use by a monitoring user.

The system apparatus or assembly that can determine and provide data about a subject to the monitoring program includes a fluid bladder against which the subject rests. A pump is in fluid communication with the fluid bladder, and the pump is operable to increase a fluid pressure within the fluid bladder. A pressure sensor can be used to monitor a subject at rest on the bladder. The pressure sensor is in fluid communication with the fluid bladder, and the sensor is operative to determine a pressure within the fluid bladder. A processing unit is configured to determine at least one parameter of the subject based on the pressure within the fluid bladder, the data comprising at least one parameter. A data base can store historical data including the pressure within the fluid bladder as raw data and one or more parameters about the subject that comprise the data. A processor can determine a quality correlation between the pressure within the fluid bladder and the at least parameter based on the data and historical data. A software program displays the data in the desired form, enabling the monitoring user to see detailed information about the subject who is using the fluid bladder.

A monitoring system can be used to detect and measure one or more parameters of a subject 5, a subject being a person or an animal. For example, a monitoring system having both a monitoring apparatus 10 and an external monitor device 26 can be used with a bed frame 11 and mattress 12, with the apparatus 10 comprising a fluid bladder 16, a pump 14, and a processing unit 15 as shown in FIGS. 1 and 2. The system apparatus 10 can additionally include a padding layer 13 on top of and/or beneath the fluid bladder 16 as shown in FIG. 1. The padding layers 13 can include one or more of a foam pad, a box spring, an additional fluid bladder, a straw-filled pad, a feather-filled pad, a sawdust-filled pad, a spring-based pad, and/or another type of padding that offers flexibility and/or softness. Alternatively, the fluid bladder 16 can be sized for use in a chair, hospital bed, crib, or another structure that regularly supports an individual subject at rest.

The bladder 16 can hold air or another fluid, such as water. The fluid bladder 16 can be sized to have a surface area nearly as large as a surface area of a top side of the mattress 12, such as a king-size, queen-size, full, twin, or other sized mattress 12, to allow the detection of a user's vital signs regardless of the position of the user. Alternatively, the bladder 16 can have a smaller size so long as it is sufficient to cover a user's heart and/or lungs. For example, a size covering an area of the mattress 12 above which the user's heart and/or lungs are expected to be positioned (e.g., a one foot by one foot square for an adult user) can be used. Incident pressure waves caused by shifting body weight in response to cardiopulmonary activity induce a change in the measured pressure. The pressure in the fluid bladder 16 can vary depending on the amount of fluid in the bladder 16, whether a user is lying on the bladder 16, the heart rate of a user lying on the bladder 16, the respiration rate of a user lying on the bladder 16, and other movement of a user lying on the bladder 16 (e.g., rolling or limb movement).

The pump 14 can be a separate unit from the bladder 16 and can be fluidly coupled to an air inlet 17 of the bladder 16 via a hose 18 as shown in FIG. 1. The pump 14 can alternatively be integral with the bladder 16 such that the pump 14 can output high pressure fluid directly into the bladder 16 instead of through the hose 18. The pump 14 can be a rotary type pump or another type of pump. The pump 14 can include an electric line 20 for connection to a power source, or the pump 14 can include a self-contained power source, such as one or more batteries. The pump 14 can also include a data line for communication with the processing unit 15.

As shown in FIG. 1, the pump 14 can be packaged with a sensor 22. That is, the pump 14 and sensor 22 can be part of an integral unit. For example, a pump housing 19 that acts as a casing containing components of the pump 14 can also contain the sensor 22. The sensor 22 can be positioned within the pump housing 19 to detect an amount of air pressure in the hose 18. For example, the sensor 22 can be positioned in a portion of the pump 14 in communication with the hose 18, such as in fluid communication with the pressurized fluid outlet 28 of the pump 14 as shown in FIG. 1. Since the hose 18 can be in fluid communication with the bladder 16, the air pressure detected by the sensor 22 can indicate the air pressure in the bladder 16. While operation of the pump 14 may affect the pressure detected by the sensor 22, the pump 14 can operate only as required to maintain an average pressure within the bladder 16 (e.g., to replace any fluid that seeps out of the bladder 16).

Alternatively, the sensor 22 can also be located separate from the pump 14. The sensor 22 can be positioned such that the sensor 22 has a sensing side within the bladder 16 and a reference side outside of the bladder 16. In this case, the sensor 22 can be positioned in the seam of the bladder 16. The sensor 22 can include a semiconductor pressure sensor or another type of pressure sensor. Additionally, other types of sensors, such as a temperature sensor, can also be included. The sensor 22 can output a pressure signal α to the processing unit 15. The sensor 22 can be hard-wired to the processing unit 15, the sensor 22 can wirelessly communication with the processing unit 15 by way of a transmitter using, for example, a standard wireless protocol (e.g., IEEE 802.11, RF, Bluetooth, or 3G), or the sensor 22 can otherwise be coupled to the processing unit 15 for communication therewith.

A controller 24, which can be a microprocessor or another device, can receive a signal indicating the pressure of the bladder 16 from the processing unit 15 to control the pump 14 as shown in the flow diagram of FIG. 2 to operate the pump 14 to maintain or increase the pressure in the bladder 16. The controller 24 can also be in communication with an air release valve or other structure for releasing air from the bladder 16 such that the controller 24 can provide an instruction to decrease the fluid pressure in the bladder 16.

It is also contemplated herein that a self-inflating bladder can be used, thereby eliminating the need for the pump. Examples of self-inflating bladders that can be used with the embodiments herein include but are not limited to those disclosed in U.S. Pat. No. 6,651,277 to Marson and U.S. Pat. No. 4,624,877 to Lea et al., both incorporated in their entirety by reference.

The processing unit 15 can comprise one or more processors, digital signal processors, and different types of memory. Other peripheral devices may be used in addition to or in the place of the depicted hardware, which is not meant to imply limitations with respect to the embodiments. In addition to being able to be implemented on a variety of hardware platforms, the processing unit 15 can be a variety of software environments, such as various operating systems. The processing unit 15 maybe implemented in any electronic device, such as a desktop or laptop computer, a handheld or portable computer-like device and other electronic media players, cellular telephones, etc.

The processing unit 15 can analyze the pressure signal α and convert it to one or more parameters of the subject. These parameters, or data, include, but are not limited to, the following examples: heart rate, respiration rate, and changes in static pressure. These parameters can be analyzed to determine one or more of the following: length of sleep, quality of sleep, position, presence or absence in bed, blood pressure, tossing and turning movements, rolling movements, limb movements, weight and/or other data. More specifically, when a subject rests against the bladder 16, each of the subject's heart beats, breaths, and other movements can create a force on the bladder 16 that is transmitted to the sensor 22. As a result of the force input to the bladder 16 from the subject's movement, a wave will propagate through the bladder 16. The sensor 22 can detect the wave, transmitting the wave as a pressure signal α that can be computed into data such as a heart rate, respiratory rate, or other parameter regarding the subject.

The ability to detect heart beat and respiration, and changes in heart beat and respiration, in combination with changes in static pressure enables a user to gather detailed information about a subject. When only a change in pressure is detected, it is unknown from the signal alone what generated the change in pressure. For example, a suitcase may have been placed on the bladder. There is no way to verify that the signal is generated from the actual subject. The systems herein can develop a unique biological fingerprint for an individual with the combination of heart beat, respiration and weight, or static pressure. For example, one subject will generate a specific static pressure on the bladder due to the subject's weight. The subject also has a unique heart beat and respiration combination. With this data, the system can determine if that particular subject is resting on the bladder. In addition, the system herein can determine changes in position of the subject by the strength of each signal in combination with the other signals. For example, a subject is lying on his back if both of the heart beat and respiration signals are strong, and lying on his right side if the heart beat signal is weak while the respiration remains strong.

The processing unit 15 receives the signal α from the sensor 22 and can perform a pattern recognition algorithm or other calculation based on the amplified and filtered pressure signal α to determine the subject's heart rate, respiratory rate and derive other biosignal properties. For example, the algorithm or calculation can be based on assumptions that a heart rate portion of the signal α has a fundamental frequency in the range of 0.5-4.0 Hz and that a respiration rate portion of the signal α has a fundamental frequency in the range of less than 1 Hz. The processing unit 15 can also receive signals from other sensors (e.g., a temperature sensor). One processing unit 15 can be used to receive signals from a plurality of sensors in the same bladder, a plurality of sensors where some are inside and some are outside of the bladder, or a plurality of sensors from different bladders in use by different subjects.

The processing unit 15 can send status signals β indicating the parameters of the subject (e.g., heart rate and respiratory rate) to an external device 26 accessible to the user, such as a text messaging platform, data logger, printer, alarm system, alert siren, or other data acquisition or actuating device; or a computer (i.e., microprocessor) capable of performing analytical functions. These are non-limiting examples and other external devices 26 know to those skilled in the art can be used. The processing unit 15 can include a transmitter to relay the status signal β to the external device 26. The transmitter can be a wireless transmitter operating using a standard wireless protocol (e.g., IEEE 802.11, RF, Bluetooth, or 3G) or can alternatively be hardwired using a phone line, Ethernet line, or other connection.

Medical facilities such as hospitals, nursing homes and psychiatric institutions can use the monitoring system for many different reasons. A medical facility might use the monitoring system apparatus 10 in each bed, or each bed of a ward or floor, with each subject or patient associated with a system apparatus 10 or bed, providing instant information to the user of what patients are in bed and what beds are available to new patients. Because the system apparatus 10 is a non-contact monitoring system, patients need not be “hooked up” to the apparatus. The system does not need to be turned on by an employee or otherwise initiated. The system can begin to monitor the patient as soon as the patient rests against the bladder 16.

Medical facilities can use the system to continuously monitor vital signs of patients while the patient is in bed. The vital signs can be sent to an external device for monitoring by a doctor or nurse. However, there are many other uses for the apparatus 10 by medical facilities. Non-limiting examples are provided herein.

One embodiment of the monitoring system and method of use is for monitoring patients with dementia. A monitoring apparatus 10 as described is used for each patient being monitored. As illustrated in FIG. 3, the sensor 22 or sensors within the apparatus 10 sense incident pressure waves within the bladder in step S1 generated by the patient on the bladder 16. The sensor 22 generates signals a representing the pressure waves and sends to processor 15 in step S2. The processor 15 analyzes signals a and generates an individual fingerprint θ for the patient assigned to that particular monitoring apparatus 10, or bed in which that apparatus is used, in step S3. Once the fingerprint θ is developed, the processor 15 can determine when that particular person is in his or her bed. In step S4, the processor 15 generates a signal β indicative of whether or not the patient is in bed based on the patient's fingerprint θ. The signal β is then sent to the external device 26 accessible to the user, such as hospital or nursing home personnel, to monitor whether a patient with dementia is in bed or out of bed. Because of the individual biological fingerprint θ, the user can also determine if the patient is in someone else's bed. The system can be used to provide an alarm when a patient gets out of bed, alerting the staff or caregiver that the patient needs supervision.

For all patients, whether with dementia or not, an alert as to when a patient is out of bed can reduce the potential for falls, a problem medical facilities work to reduce. Supervision can be provided to a patient when the user receives an indication that a patient's heart rate and respiration are no longer sensed. This can be an indication that the patient has sat up and is preparing to get out of bed. Being able to provide assistance to patients as they get out of bed can reduce the number of slip and fall accidents in a medical facility setting.

The data generated by the processor 15 in the monitoring system described in FIG. 3 can also provide guidance for staffing requirements. This guidance may be particularly useful to determine nighttime staffing requirements. For example, in an institution or medical facility with long term care, the monitoring system can be used to track the number of times each patient gets out of bed by tracking the combination of static pressure, heart rate and/or respiration. The length of time the patient stays out of bed, as well as the overall length of time the patient sleeps, can also be tracked with the same data. This data can be stored and used to ascertain the average activity of the patients during the nighttime. The activity of the patients during the nighttime hours can determine the amount of staffing required to sufficiently provide for the safety and well being of the patients.

The monitoring system can also be used to monitor a patient's time in bed and movement in bed. Another embodiment of the monitoring system and method of use is for the prevention of bed sores, also called pressure ulcers. Conventional prevention of bed sores is attained by moving patients based on a schedule of time periods. Some methods sense pressure changes in a pad or plate. When only pressure is monitored, it is difficult to distinguish the extent of movement of the subject. For example, a light weight subject may completely turn over, generating incident waves of strength A. A subject with more weight may only move a limb and generate incident waves of the same strength A. The monitoring system as disclosed can detect heart rate and respiration, as well as static pressure changes in the bladder 16. When a subject simply moves a limb, the strength of the heart beat and respiration signal do not change, while the static pressure does change. However, when a subject rolls over onto his side, the strength of at least one of the heart beat and respiration decreases, along with the change in static pressure. Because heart rate and respiration are still detected, it is clear the subject is still in bed. However, the change in the strength of the heart beat and/or respiration indicates that the subject has changed position enough such that he does not need to be rolled over for bed sore prevention.

As shown in FIG. 4, the sensor 22 detects heart rate, respiration and static pressure and generates a signal α that is sent to the processor 15 in step S10. The processor 15 analyzes the signals sent by the sensor 22. If the processor 15 determines that there has been a change in static pressure while heart beat and respiration remain consistent, as in Step 12, the patient has moved, but not sufficiently to prevent a pressure ulcer. Accordingly, the patient remains on the list of those requiring turning over by staff, as in step S14. If the processor 15 determines that there has been a change in static pressure and a change in the strength of the heart beat and/or respiration, as in Step 16, the patient has turned over sufficiently to prevent a pressure ulcer. Accordingly, the patient is noted as not requiring turning over by the staff. The time period within which a patient is turned over is restarted.

Medical facilities and sleep clinics can monitor patients sleep without the need for electrodes contacting the patient, for use in determining a subject's sleep quotient and particular sleeping patterns of individual patients. For example, sleep apnea or restless leg syndrome can be diagnosed with the system. Sleep apnea can be diagnosed using heart rate, respiration and static pressure. Sleep apnea in a subject can be indicated by a change in static pressure and heart rate while respiration ceases. Restless leg syndrome can be diagnosed by frequent changes in static pressure without a change in heart rate and respiration.

The unique biological fingerprint θ associated with an individual can be used to monitor the compliance of regulations by, for example, prison release programs and half way houses. These programs have regulations to which participants must comply in order to remain in the program. As non-limiting examples, participants may be required to be in bed from 11 pm to 7 am, and participants may be required to refrain from drug use. Because the system is a non-contact monitoring system with the bladder of the system located under a mattress, there is no set up when the subject lies down or rests against the bladder. If desired, the subject need not know the monitoring system is there. However, due to the unique biological fingerprint θ that can be generated for each participant, the system cannot easily be manipulated to produce false data.

Current state fiscal crises and significant overcrowding at criminal justice facilities around the country have placed an intense focus on both community corrections and re-entry initiatives at both the state and federal levels. Such facilities to which prisoners are released can equip each bed with a monitoring apparatus 10 as described herein. Each apparatus 10 can communicate with a single processor 15 or more than one processor. The processor 15 can generate data to one or more external devices 26 that can be actively monitored by one or more users. In addition or in the alternative, the data can be saved for review at a later date. The processor 15 can determine the time in which the subject is in bed by the time the unique fingerprint θ is detected. Whether the subject remained in bed all night can be documented. Potential stress, alcohol or drug use can be detected by a change in heart beat and/or respiration. The ability to monitor compliance with a curfew, for example, without a device such as an ankle tether, can improve morale, decrease data manipulation, and reduce staffing costs.

The monitoring system can be used for many applications in a home setting. Because the monitoring apparatus 10 is a touchless system and requires little installation, there is no extensive training, preparation, or change in a subject's behavior in order to incorporate the system into regular use. A medical facility might send system apparatus 10 home with a patient upon discharge and use an on-site monitoring program described below to actively monitor that patient's parameters for a period of time following major surgery, for example. Professional home health care providers can use the system to enhance their capabilities and improve care. Non-professional care givers can use the monitoring system to gather data for physicians, set reminders for the turning of a patient or providing medication, etc. Periodic updates can be wirelessly sent to a medical professional. Professional home health care workers and non-professional caregivers can utilize the monitoring system for family members with dementia as described above.

Another embodiment of the monitoring system disclosed herein is a portable monitoring device 50 for discharged patients. Patients discharged after post cardiac trauma or surgery, for example, can be provided the portable monitoring device 50 to take home with them. The portable device 50 shown in FIG. 5 includes the bladder 16, which can roll up into a portable size, the pump 14, the sensor 22, and the processor 15. The discharged patient can unfold the bladder 16 and place it under a mattress. The pump 14 can be an electrical pump that simply requires plugging in to operate to fill up the bladder 16 to the required pressure with fluid, such as air. The sensor 22 can be already permanently provided in the bladder 16, so that the discharged patient need not do anything with the sensor 22. The processor 15 can be a small unit that can just be placed on a night stand or the floor. The processor 15 can communicate with an external device 26 wirelessly, or a USB device can be used to collect data which is later brought by the patient or a caregiver for downloading by medical personal for reviewing. If the data is transmitted wirelessly, the physician has the benefit of accessing real time vital statistics of the discharged patient. The stored data can be trended over time as desired.

An individual can utilize an embodiment of the monitoring system for many reasons, such as to monitor sleep patterns, monitor overall health, monitor health during physical training, and monitor health during dieting. The data gathered by the individual can alert him or his doctor of sleep issues based on long-term sleep trends, such as sleep apnea or restless leg syndrome. The data can provide other health information such as the subject's fitness level, cardiovascular condition, etc. The data can simply be stored in a data base for use in the future in the event a medical condition or event occurs.

An individual can use the portable monitoring device 50 or an apparatus 10 for use in his or her home for at least the reasons discussed. If the individual is monitoring his or her sleep patterns, the user would set up the fluid bladder 16 of the apparatus 10 in his bed under the mattress 12 as described above. If the individual is monitoring his vital signs only, the individual may choose to position the bladder 16 of the apparatus 10 in a chair 60 as shown in FIG. 6 rather than a bed so that the system monitors the user's vital signs when he is sitting. The individual may, for example, work out and periodically sit in the chair against the bladder 16 to get a reading of his heart rate or respiration at desired intervals. The bladder 16 can be positioned on both the seat of the chair and the back of the chair to better read both weight and heart beat and respiration. It is also contemplated that the bladder 16 can only be located on the back of the chair 60 to monitor heart rate and respiration of the subject.

A parent or caregiver can use the monitoring system to monitor a baby's vital signs and movement while the baby is in a crib. The bladder 16 of the apparatus 10 can be placed under the crib mattress. An alarm can sound, for example, when the infant's breathing has stopped, alerting the parents to the need for intervention. A parent or caregiver may also use the monitoring system on children, perhaps through a physician, to determine if the child is getting sufficient sleep or if a lack of sleep is contributing to other behavioral manifestations.

The applications described are not meant to be limiting. Other applications consistent with the scope of the disclosure are contemplated. Each of the applications can be used with an external monitor device 26 as noted. One such external device 26 can be a monitoring program 100 as described herein.

The monitoring program 100 is shown in FIG. 7 with the monitoring apparatus 10. The monitoring program 100 can comprise a monitoring software program 30 (MSP), a display 32, a user interface 34 and a database 36. The monitoring software program (MSP) 30 can be locally-installed on a computer, be located on a separate computer server and connected to a local computer through, for example, a private access connection (e.g. local area network), or be an application embedded in and accessible through a web server via the Internet. The computer can be a laptop computer, a desktop computer, a workstation, a handheld device, a server, a cluster of computers or other any suitable computing device. The MSP 30 can also be integrated into the processing unit 15. The database 36 can store historical status signals β for one or a multiple of subjects and can provide the historical data to the MSP 30.

Signals β representing the data may be combined with additional signals or data (e.g. generated by one or more additional sensors), filtered and relayed to the MSP 30 for generating content and displaying the content to a user. Additional sensors can include temperature sensors, light sensors, weight sensors, audio sensors, video, etc. The MSP 30 can generate content from the data (one or more parameters) as desired or required by the user and display the content associated with a particular subject through different screens on the display 32. The MSP 30 and display 32 can be connected to the user interface 34. The user interface 34 can include various user devices, such as a keyboard and mouse, a touch screen, stylus, microphone, etc.

The following description of displays in the monitoring program 100 and related interfaces illustrate exemplary content and screens generated by the MSP 30. The description and related figures reference use of the software program to generate content and display various parameters of subjects for use by various monitoring users. However, the references are merely exemplary and are not to limit the scope of embodiments of the invention. Those skilled in the art will realize that other embodiments may implement the MSP in the context of other areas.

The MSP 30 described herein can generate and display a log-in screen 110 as seen in FIG. 8. The log-in screen can prompt a user to enter a previously registered user name 112 and password 114. In addition, a user can select a sign-in 116 option for new users to register to use the monitoring program. Registration can include the association of a subject and the subject's monitoring apparatus 10, such as a sensor ID, with the monitoring program 100. A user can be any person who is monitoring the subject or subjects for any reason. As non-limiting examples, the user could be the actual subject, medical facility personnel, home healthcare workers, family or friends caring for the subject, nursing homes, sleep clinics, trainers, prison discharge program operators, etc.

The MSP 30 can generate default content and use default displays that are preprogrammed for ease of use. The MSP 30 can also be modified by the user through the user interface 34 to generate and display content selected by the user. For example, in any of the display screens described herein, a default screen can include all of the options described, while a modified user-specific screen may display only the content in which the user is interested. Different users will be interested in different content. Examples of different uses will be described herein, illustrating how different users may be interested in different data and content.

The MSP 30 can generate and display a home page 120 that provides an overview 122 of each monitoring system apparatus 10 available to the user to monitor, as shown in FIG. 9. For example, if a user is also the subject, the user may only see his self or herself listed on the home page 120. If the user is a parent, the user could see one or more children listed on the home page 120. If the user is a medical facility, the user may see any number of monitoring apparatus 10 listed by one or more of subject name, bed number and room number, for example. FIG. 9 is exemplary and illustrates a user monitoring two subjects each using a monitoring system apparatus 10 available to this user and identified by the subjects' names.

The home page 120 gives a brief overview 122 of each monitoring system 122 for which the user is registered to view. The home page 120 can provide information regarding one or more subjects with a quick review of the screen. The overview can indicate if the monitoring apparatus 10 is in active use 124, meaning that the subject is at rest against the bladder. As used in the figures, the symbol of a bed is used, with active use indicated by a subject on the bed and inactivity or non-use indicated by the bed without a subject. The bed is used as an example only. The system can be used in chairs to monitor a subject when the person is sitting if desired. If the monitoring apparatus 10 is active, the real time heart rate 126 and/or respiration 128 of the subject can be shown. The length of time 130 the subject has been being monitored can be included, which can indicate the amount of time the subject has been in bed. Any messages 132 entered by one of the users through the user interface 34 can also be generated and displayed. The home pate 120 can also include whether the monitoring apparatus 10 is currently connected or disconnected 134, with disconnected systems indicating availability, for example, if the system is used in a hospital setting. The home page 120, as any of the pages, can be set up to display what is desired or required by a specific situation or user. The home page 120, as well as the other screens discussed herein, can have shortcuts 140 to quickly go to one of the other screens.

The home page 120, as well as any other page, can include an alarm indication 136. Alarms or alerts may be triggered by data that is outside a predetermined value range or meets pre-selected user trigger points. Simple alarms or alerts are audible and/or visible signals, such as horns, buzzers, sirens, lights, and the like. Alarms or alerts may be sent to pre-selected health care professionals (including paramedics, physicians, nurses, police, and the like), relatives and/or guardians, public health agencies, child services, etc., as determined by the user. Alarms, data messages and/or alerts may also be localized to particular places in a home, hospital, elderly, care facility, or infant care facility. Such signals may be transmitted by wired or wireless technology, such as cabling, WiFi, Zigbee, Bluetooth, etc., for contacting receiving devices such as cell phones or personal digital assistants (PDAs).

The MSP 30 can also generate content and display a dashboard 150, as shown in FIG. 10. The dashboard 150 is a detailed look at the data monitored by the user. If more than one subject or monitoring system 10 is listed in the overview 122 seen by the user, the user can select a subject or system 10 from the overview 122 on the home page 120, pulling up the dash board 150 for that particular subject. As illustrated in FIG. 10, the dashboard 150 can include information about a current or recent monitoring session, which can be a night's sleep, for example. Content such as the time the subject is asleep while being monitored, or sleep time 152 of the subject, can be generated and displayed, as well as amount of movement 154 of subject during the session, and average heart rate 156 and breathing rate 158 during the session. This content can be displayed in bar graph style as illustrated, with each data parameter shown against a scale 160 indicating whether or not the parameter is within a predetermined range. The predetermined range can be a default range, a normal or desirable value that is based on the subject's age, weight, sex, etc. and determined by a physician for example, or set based on governmental physical fitness standards. The predetermined range can also be set by the user through the user interface 34.

The dashboard 150 can also display the subject's sleep quotient 162 based on the subject's heart rate, respiratory rate, amount of time spend in REM sleep, total time in bed, and other considerations. A summary 164 can be provided of sleep patterns for the current session, as well as a historical average.

An interactive calendar 166 can be provided on the dashboard 150 that can provide an overview of a week or a month at one time. The user can select an earlier date to receive a historical overview of a particular data parameter, such as the sleep quotient. Selecting a specific date in the calendar 166 can bring the user to the dashboard 150 and other screens for the particular date selected. The days of the calendar 166 can be color coded to represent particular data as desired, such as sleep quotient, to quickly indicate to the user how frequently the subject's data is within the predetermined range. For example, dates in which the subject's sleep quotient is within range may be shaded green while dates in which the sleep quotient is out of the predetermined range may be in orange.

The dashboard 150 can also include a diary 168 in which the user can record incidents that may have bearing on why one or more parameters had a particular value that session. For example, a user may record environmental factors that influence sleep or vital signs, such as caffeine intake, alcohol intake, exercise performed, etc. The diary can provide the user information to analyze a particular pattern that develops. The dashboard 150 can also include a timeline 170 for the current monitoring session.

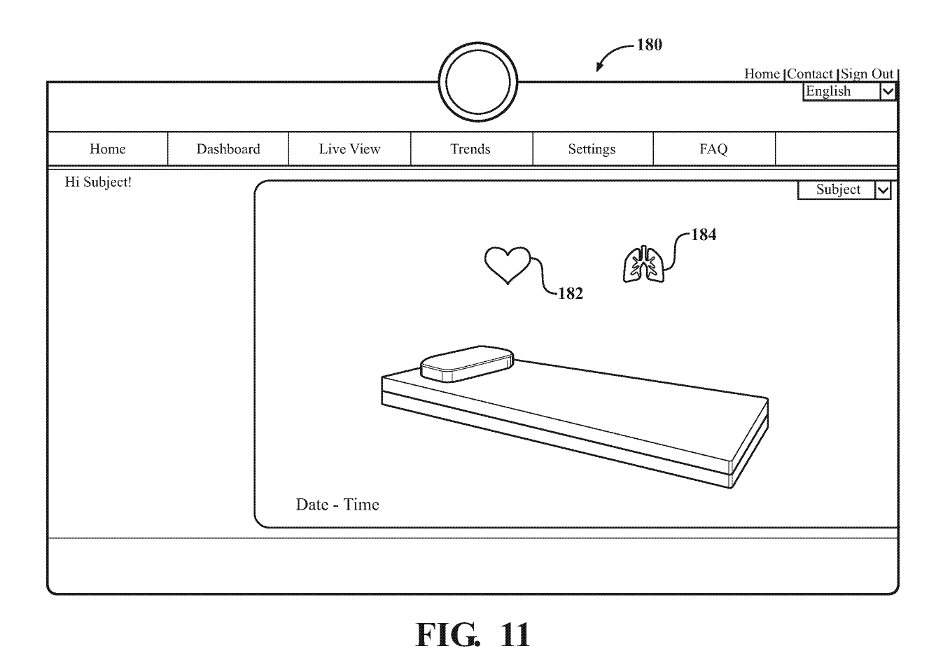

FIG. 11 illustrates a live view 180 screen generated and displayed by the MSP 30. The live view 180 can be a real time view of a particular subject's heart rate 182 and respiration 184, for example. The live view can also indicate whether the subject is currently asleep. The live view can also incorporate a live video stream of the subject. The date and time can also be displayed.

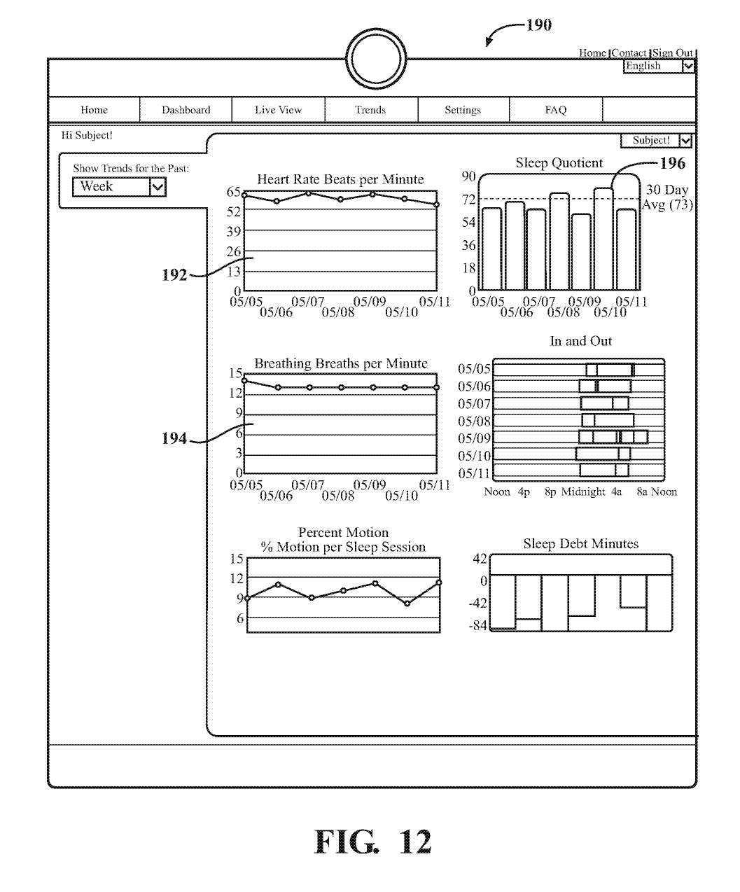

FIG. 12 illustrates a trends screen 190. As noted earlier, raw pressure values and data can be saved in the data base 36. The trends screen 190 provides graphical representations of the historical data from the data base 36 for one or more of the monitored parameters based on the default content generated or the content generated based on modifications of the user. For example, the trend screen 190 can display a line graph of the subject's hear rate 192 or respiration 194 for a predetermined period of time, such as a day, week or month. A bar graph can display a subject's average daily sleep quotient 196 for a selected period of time. Other trends can be displayed as desired or required by the user.

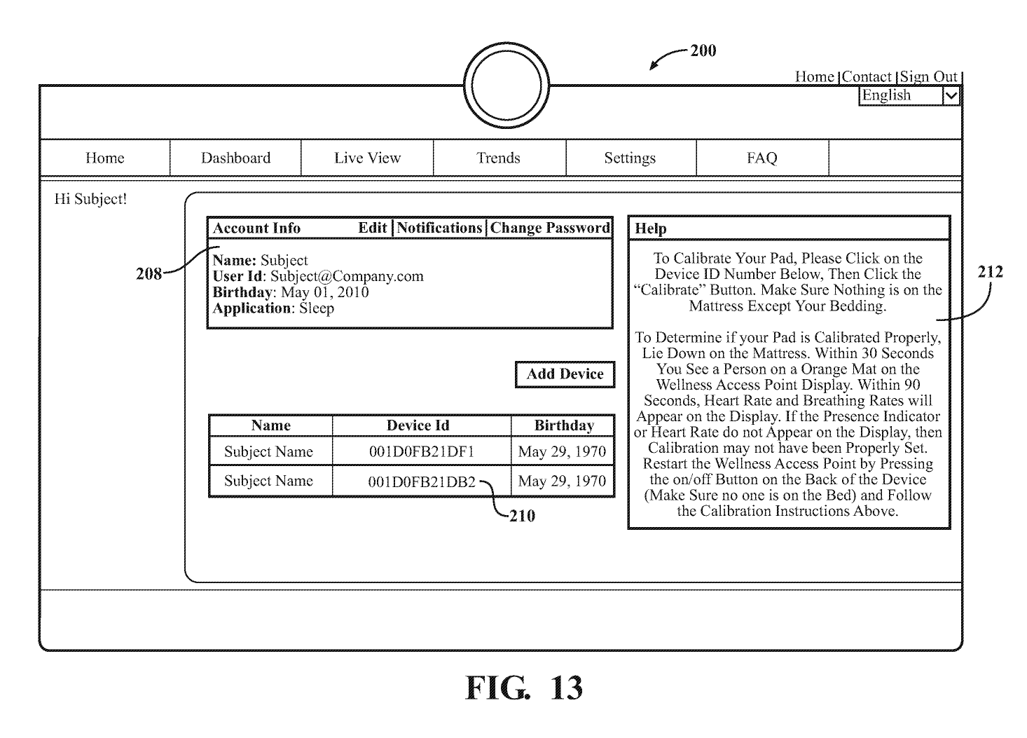

FIG. 13 illustrates a settings screen 200. The settings screen 200 can include account information 208, monitoring apparatus identification 210, such as serial number, calibration alerts or logs, etc. Particular screen setup information can also be entered in the settings screen 200, such as what data and trends the user would like displayed on the trend screen 190 or what information the user would like displayed on the dashboard 150. Predetermined periods can be set for displaying historical data, etc. For each subject, information such as weight, age, sex, etc. can be entered. The settings page can also include one or more help screens 212.

Any of the screens disclosed herein can include the capability to print the screen or the information displayed, download current and historical data, delete the information, etc. Furthermore, the MSP 30 is not limited to the particular displays herein. Other screen displays are contemplated that are within those skilled in the art and within the spirit and scope of the disclosure.

Following are non-limiting examples of how the monitoring system, including the apparatus 10 and monitoring program 100, can be used by various subjects and users. Other uses and users that are within the scope and spirit of the disclosure are contemplated.

As noted, the MSP 30 can be used to generate content and display for a plurality of system apparatus 10. A medical facility might use the monitoring system apparatus 10 in each bed, or each bed of a ward or floor, and use a single monitoring program 100 to monitor each of the beds with the MSP 30 generating and displaying content for each monitoring apparatus 10 on one or more displays 32. Each subject associated with a system apparatus 10 would be displayed on the home page 120, providing instant information to the user of what patients are in bed and what beds are available to new patients.

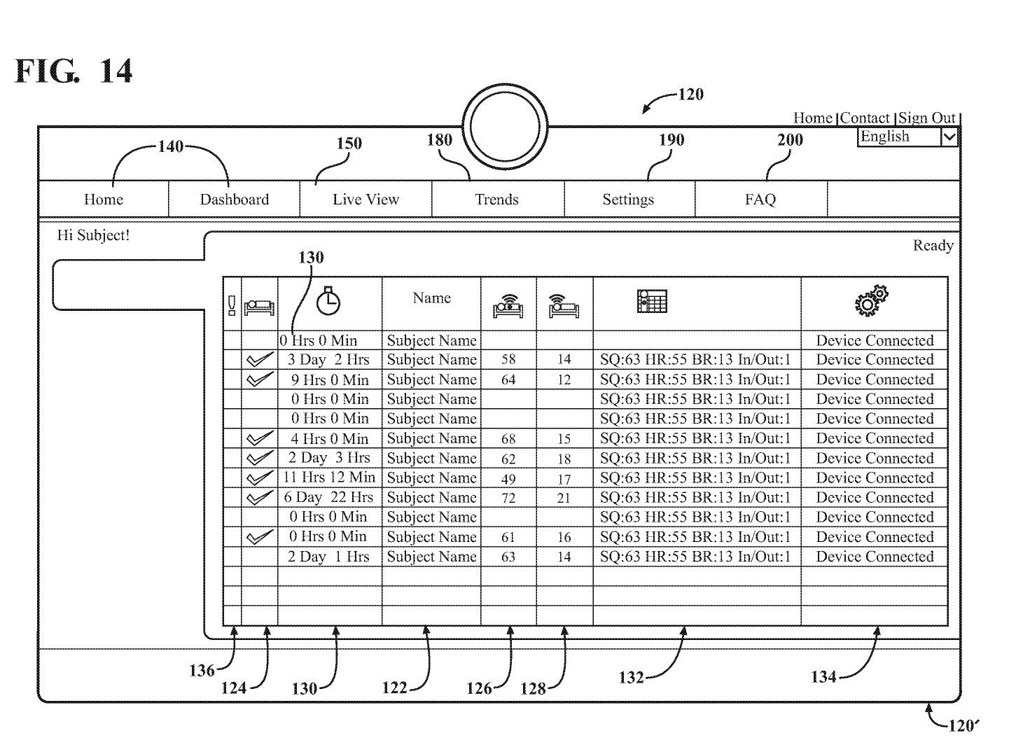

The monitoring system can be used to continually monitor the patients' vital signs. FIG. 14 is an example of a home page 120 that provides an instant view of the heart rate 126 and respiration 128 of a large number of patients. The data is real time data, as it can be updated at predetermined intervals such as every ten seconds. As mentioned earlier, an alarm indicator 136 can be included on the home page 120. The alarm indicator 136 can signal to a user when a patient is in distress, such as when the heart rate is too high or too low. The user can view the dashboard 150 of the patient by selecting the user from the overview 122 on the home page 120 to get a detailed view of the particular patient.

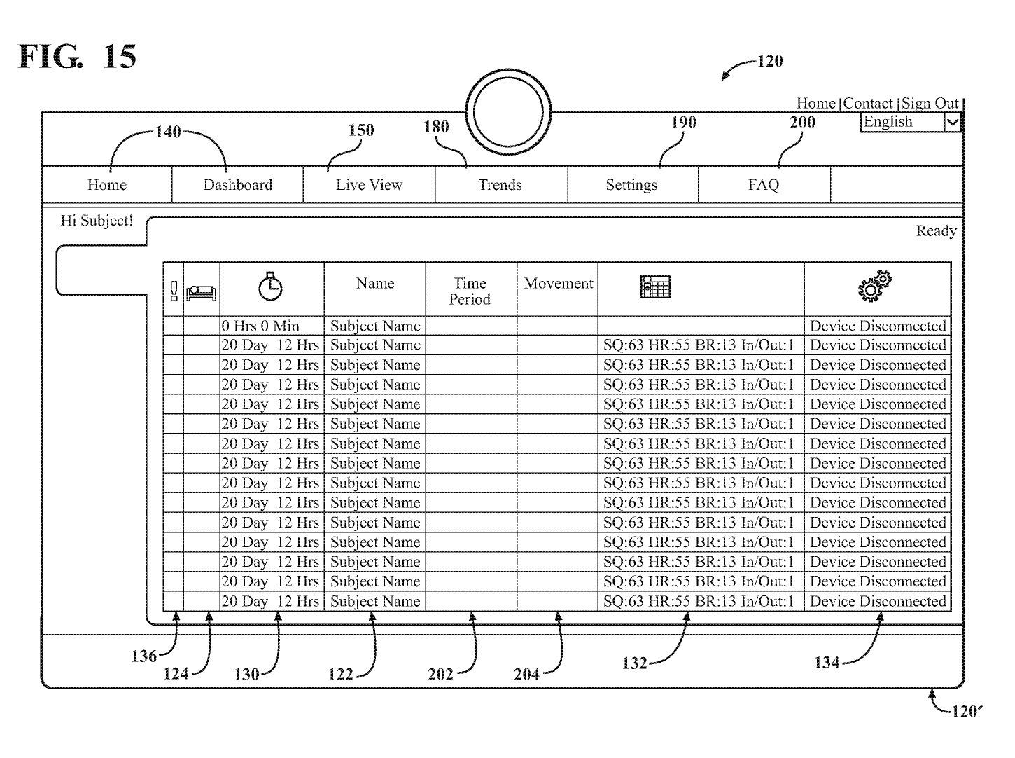

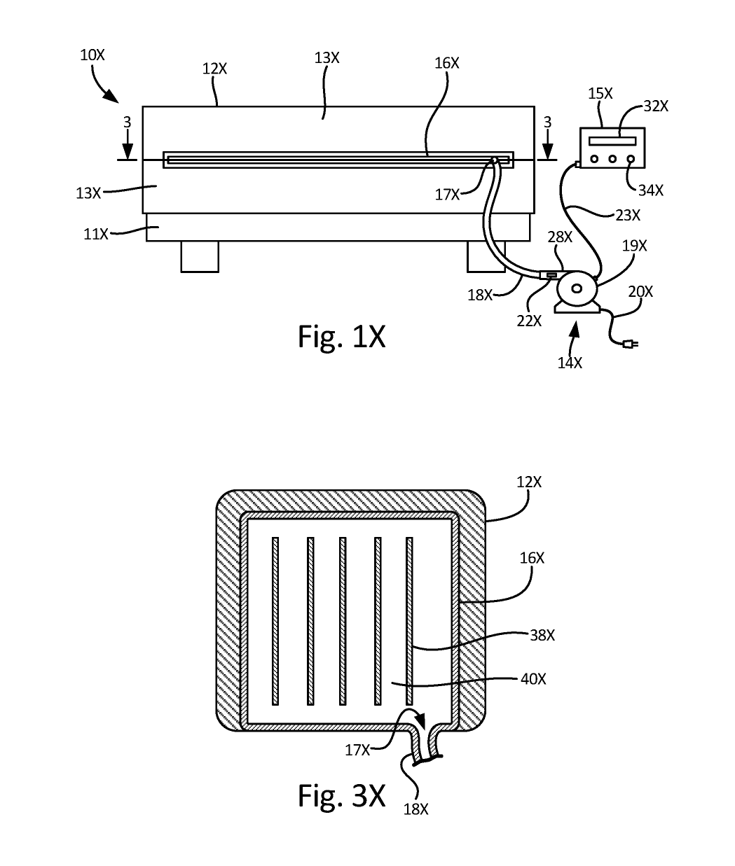

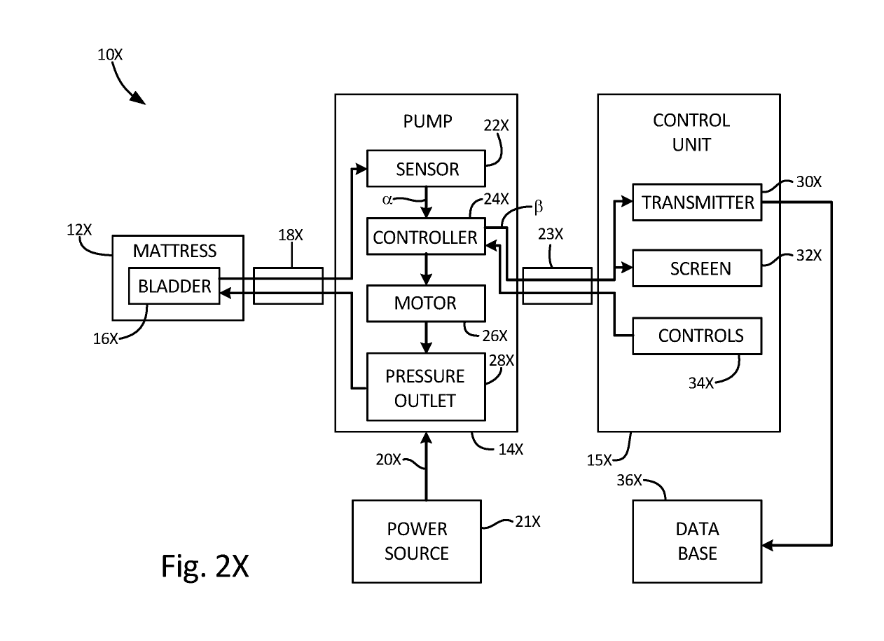

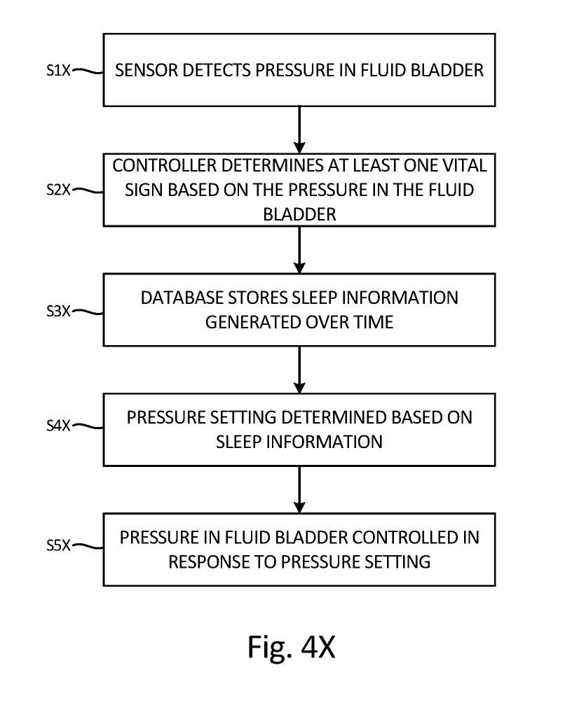

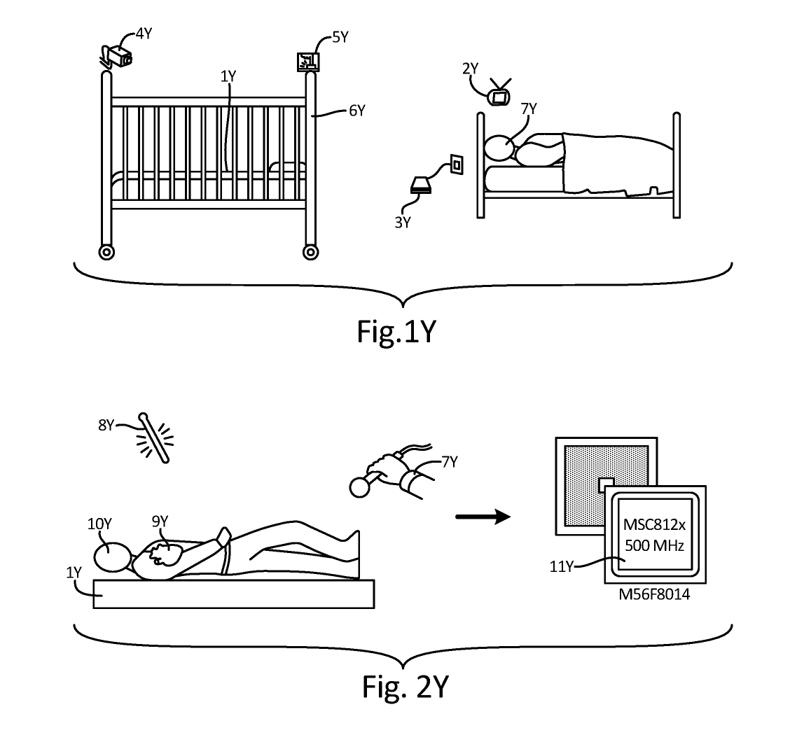

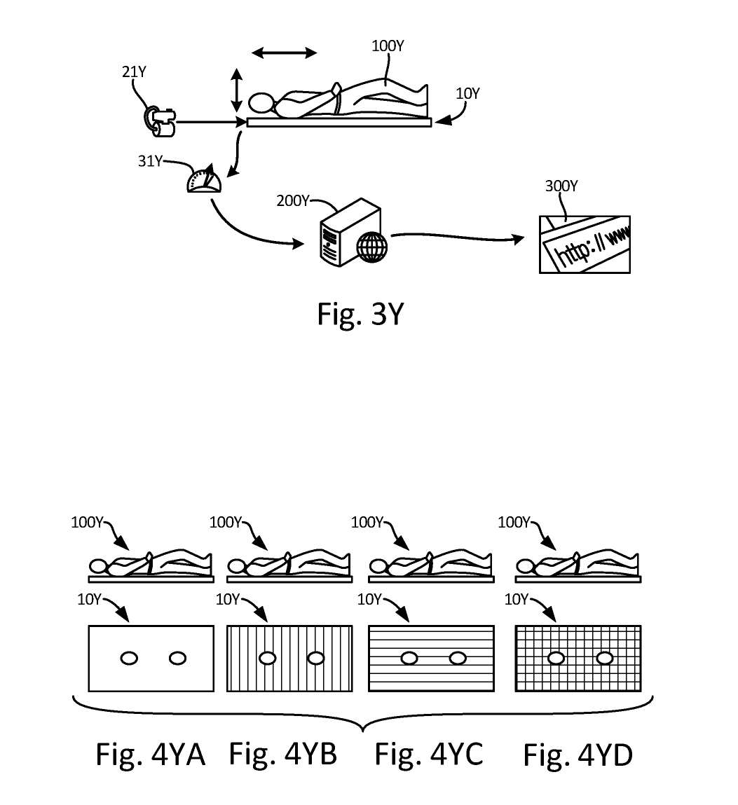

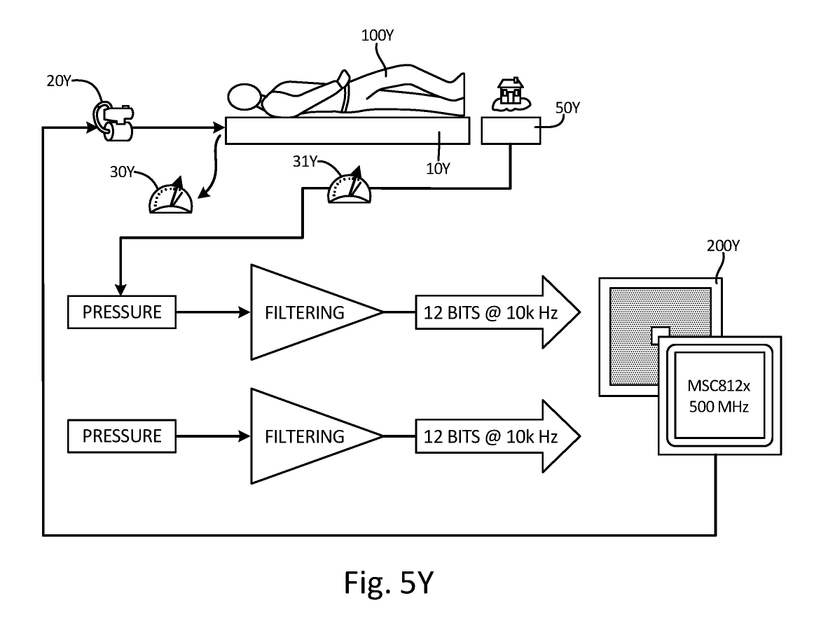

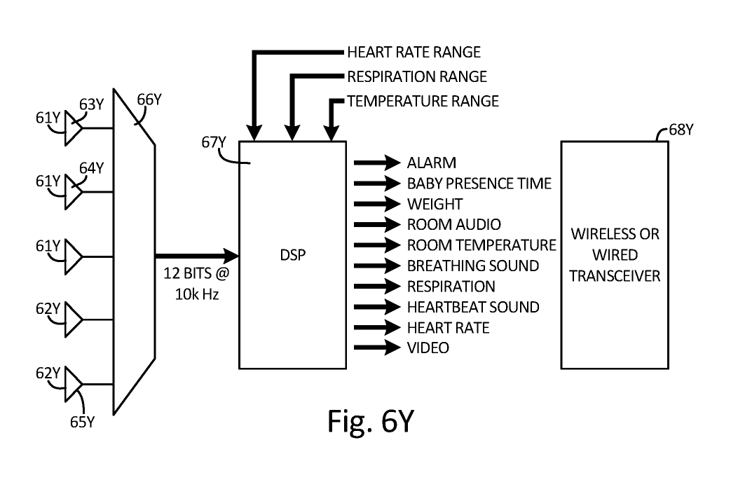

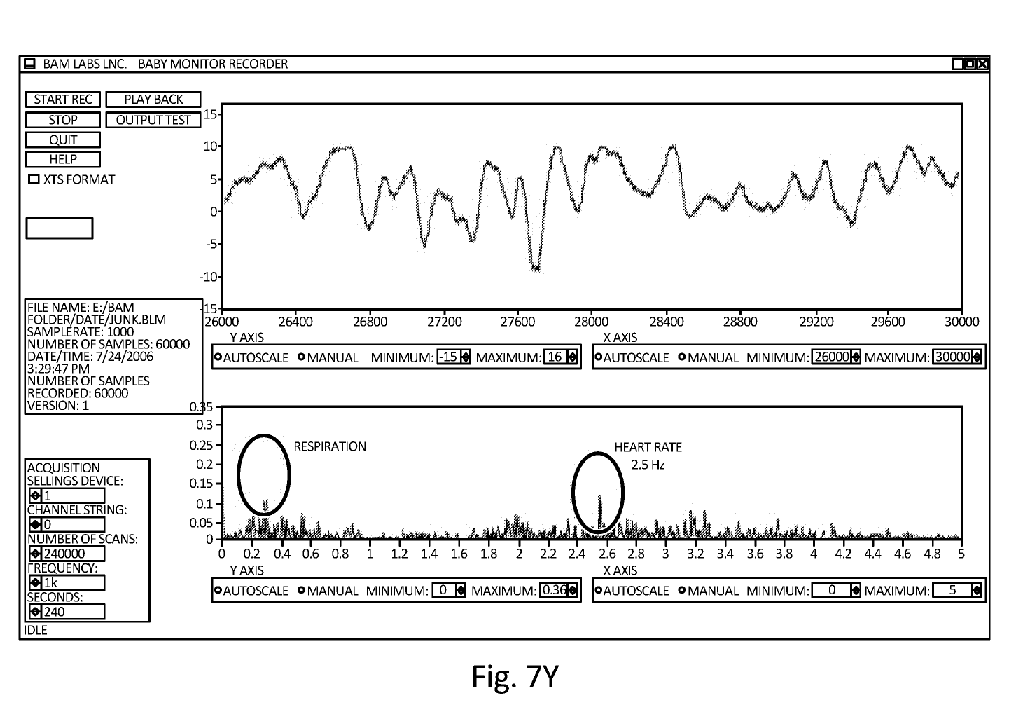

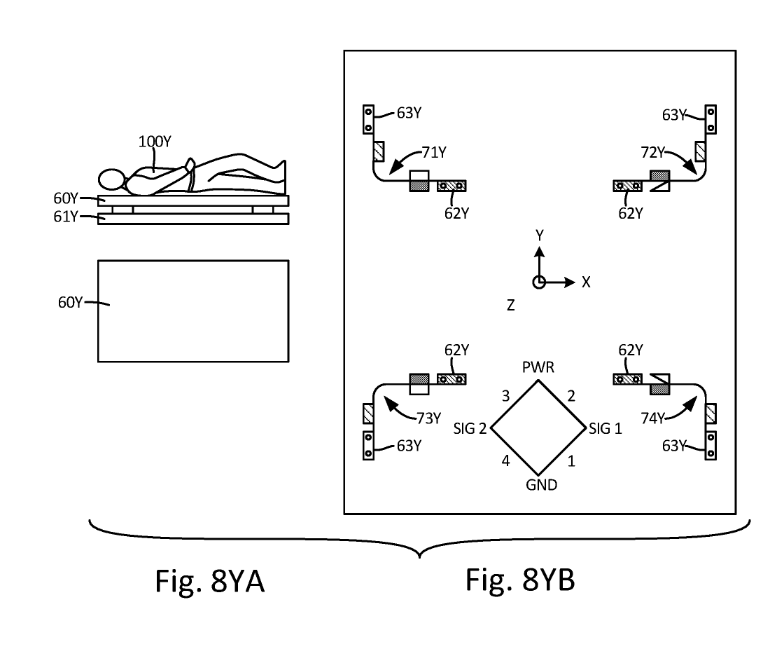

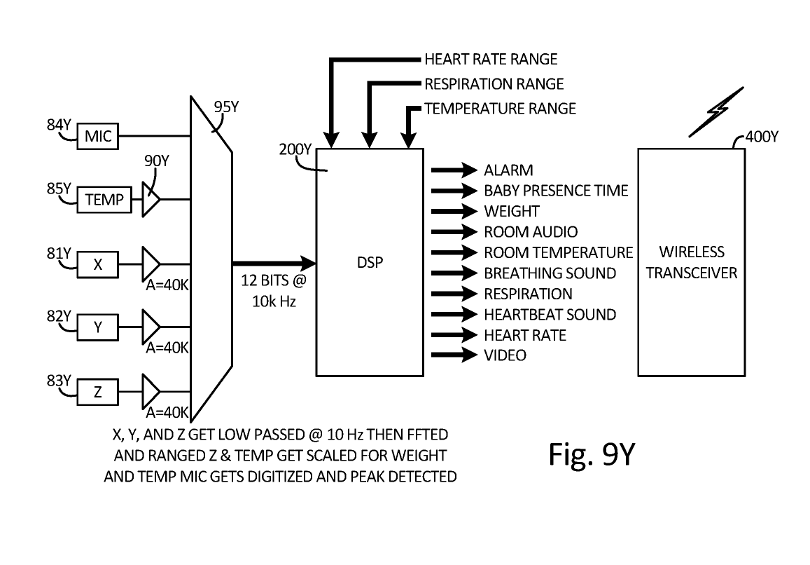

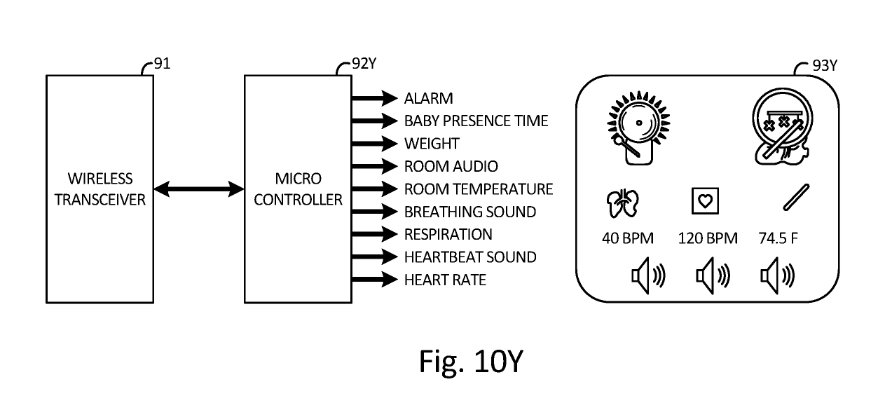

FIG. 15 is an example of a home page 120 that can be used to address the prevention of bed sores. Rather than having an instant view of heart rate and respiration, the home page 120 can provide an instant view of the length of time a patient is in bed 202 and a measurement of time between movements 204 sufficient to prevent bed sores. The alarm 136 can indicate when a patient must be turned if sufficient movement has not been detected. Messages 132 can be entered by staff to indicate, for example, when a patient was last moved by staff to indicate the frequency the staff had to assist. This can assist in tracking the amount of staff time and resources necessary to simply prevent bed sores, a typical and expensive problem in most medical facilities. The program can also monitor staff efficiency in responding to alerts, alarms or reminders for use by administration.

The monitoring system can be used by an individual subject at home, as the installation of the apparatus 10 is minimal and does not have any components that must be applied directly to the subject's body. The display 32 maybe implemented in any electronic device, such as a desktop or laptop computer, a handheld or portable computer-like device, MP3 and other electronic media players, cellular telephones, etc. The subject, also the user in this example, would register his apparatus 10 and himself on the log-in screen 110 of the monitoring program 100 to initiate communication between the apparatus 10 and the program 100. On the settings screen 200, the user would input through the user interface 34 the parameters he would like to monitor and how he would like the data displayed or use the default content and displays.

While the invention has been described in connection with what is presently considered to be the most practical example, it is to be understood that the invention is not to be limited to the disclosed example but, on the contrary, is intended to cover various modifications and equivalent arrangements included within the spirit and scope of the appended claims, which scope is to be accorded the broadest interpretation so as to encompass all such modifications and equivalent structures as is permitted under the law.

This application claims priority from pending U.S. patent application Ser. No. 11/849,051, filed Aug. 31, 2007, and U.S. Provisional Application Ser. No. 60/846,642 filed Sep. 22, 2006, each of which is incorporated herein in its entirety by reference.

The present invention pertains to a vital sign monitoring apparatus.

Historically, monitoring vital signs of a person has required expensive equipment, such as an electrocardiogram (EKG) or a ballistocardiograph (BCG). In addition to being prohibitively expensive for many situations (e.g., home use), both EKGs and BCGs can be too cumbersome for use outside of medical facilities. EKGs, for example, typically necessitate attaching electrodes to the bodies of users, while BCGs rely on large, heavy, and unaesthetic force-measuring platforms that users lie on.

In more recent times, devices including piezoelectric films or arrays of sensors have been developed to measure heart and respiration rates. A user can lie on the device, and the film or sensors can generate a signal indicate of the user's heart rate and/or respiration rate. However, these devices can also be expensive.

Some known air mattresses each include a pump connected to the respective air mattress by a hose. The pump can produce a high pressure to force air into the air mattress. However, the air mattress can lose air over time, causing the pressure in the air mattress to drop beneath a preset level. In order to reduce the problems associated with air loss, the pump can include a pressure sensor, and the pump automatically turn on when the pressure drops below the preset level. As a result, a user does not have to periodically turn on the pump to increase the air pressure in the air mattress.

A pressure sensor used to communicate with the pump can additionally be leveraged to detect vital signs, such as a heart rate and respiratory rate of a person lying on the air mattress. According to an example of a sleep monitoring system that can determine at least one vital sign of a person, the sleep monitoring system includes a fluid bladder. A pump is in fluid communication with the fluid bladder, and the pump is operable to increase a fluid pressure within the fluid bladder. A sensor is packaged with the pump. The sensor is in fluid communication with the fluid bladder, and the sensor is operative to determine a pressure within the fluid bladder. A controller is configured to determine the at least one vital sign based on the pressure within the fluid bladder.

As a result, the cost of the sleep monitoring system can be reduced compared to many vital sign monitoring devices. Further, since the sleep monitoring system can be less cumbersome to use compared to many vital sign monitoring devices, the sleep monitoring systems can be used outside of a medical center environment. Additionally, since a pump of a conventional air mattress may include a pressure sensor, that pressure sensor can be leveraged to create the sleep monitoring system by merely providing a software upgrade. Also, by analyzing sleep information generated over time, the sleep monitoring system can provide a pressure setting customized for a specific user to improve the user's sleep.

Another example of sleep monitoring system is also provided. The sleep monitoring system includes a fluid bladder. A pump is spaced from the fluid bladder, and the pump has a housing containing pump components and defining a fluid inlet for receiving fluid and a fluid outlet for outputting fluid pressurized by the pump. An elongate conduit fluidly couples the fluid outlet of the pump and the fluid bladder. The conduit provides a passage for the fluid pressurized by the pump to increase a fluid pressure within the fluid bladder. A pressure sensor is physically coupled to an interior of the pump housing such that the sensor is part of an integral pump unit, and the pressure sensor is configured to detect a pressure of fluid at the fluid outlet of the pump. A controller is configured to determine the at least one vital sign based on the pressure within the fluid bladder.

An example of sleeping pad is also provided. The pad includes a fluid bladder. A pressure sensor is in fluid communication with the fluid bladder and is operable to detect a fluid pressure within the fluid bladder. A controller is in communication with the pressure sensor and is operable to determine at least one vital sign of a user based on the pressure within the fluid bladder. A memory for storing historical data including the pressure within the fluid bladder and the at least one vital sign and a processor for determining a sleep quality correlation between the pressure within the fluid bladder and the at least one vital sign based on the historical data are also included. A pressurized fluid source is operable to increase a pressure within the fluid bladder when the sleep quality correlation indicates a sleep quality of the user would improve if the pressure within the fluid bladder were higher,

A sleep monitoring system 10X can include a mattress 12X, a pump 14X, and a control unit 15X as shown in FIGS. 1X and 2X. The mattress 12X can include a fluid bladder 16X. The mattress 12X can be sized for use on a king-size, queen-size, full, twin, or other sized bed frame 11X. The mattress 12X can additionally include a padding layer 13X on top of and/or beneath the fluid bladder 16X as shown in FIG. 1X. The padding layers 13X can include one or more of a foam pad, a box spring, an additional fluid bladder, a straw-filled pad, a feather-filled pad, a sawdust-filled pad, a spring-based pad, and/or another type of padding that offers flexibility and/or softness. Alternatively, the mattress 12X can be used sized for use in a chair, hospital bed, crib, or another structure for which padding can add comfort.

The bladder 16X can hold air or another fluid, such as water. In addition to holding air or another fluid, the bladder 16X can enclose foam or another material through which fluid waves of an expected magnitude can propagate a sufficient distance without being too dampened. The fluid bladder 16X can be sized to have a surface area nearly as large as a surface area of a top side of the mattress 12X to allow the detection of a user's vital signs regardless of the position of the user. Alternatively, the bladder 16X can have a smaller size, such as a size covering an area of the mattress 12X above which the user's heart and/or lungs are expected to be positioned (e.g., a one foot by one foot square for an adult user). Even if the user is positioned on the mattress 12X such that the user's heart and/or lungs are not directly above the bladder 16X, pressure fluctuations caused by the user may still be received by the bladder 16. The pressure in the fluid bladder 16X can vary depending on the amount of fluid in the bladder 16X, whether a user is lying on the bladder 16X, the heart rate of a user lying on the bladder 16X, the respiration rate of a user lying on the bladder 16X, other movement of a user lying on the bladder 16X (e.g., rolling or limb movement), the temperature of the fluid in the bladder 16X, and other considerations.

The pump 14X can be a separate unit from the mattress 12X and can be fluidly coupled to an air inlet 17 of the bladder 16X via a hose 18X as shown in FIGS. 1X and 2X. However, the pump 14X can alternatively be integral with the mattress 12X such that the pump 14X can output high pressure fluid directly into the bladder 16X instead of through the hose 18. The pump 14X can be a rotary type pump or another type of pump. The pump 14X can include an electric line 20X for connection to an outlet 21X as shown in FIG. 2X or for connection to another power source, and the pump 14X can also include a data line 23X for communication with the control unit 15. Alternatively, the pump 14X can include a self-contained power source, such as one or more batteries.

As shown in FIG. 2X, the pump 14X can be packaged with a sensor 22X and a controller 24X in communication with both the sensor 22X and the control unit 15X. That is, the pump 14X and sensor 22X can be part of an integral unit. For example, a pump housing 19X that acts as a casing containing components of the pump 14X can also contain the sensor 22X. The pump housing 19X can be made from a rigid material (e.g., ABS plastic, polypropylene, a metal, or another material), and the pump housing 19X in its assembled form containing components of the pump 14X and sensor 22X can have the appearance of a monolith or of a single, commercial component. Also, the pump housing 19X can define a fluid inlet 27X and a pressurized fluid outlet 28X. Fluid at an ambient pressure can be received by the pump 14X through the inlet 27X, and the pump 14X can increase the pressure of the fluid before outputting the fluid through the outlet 28X.

The sensor 22X can include a semiconductor pressure sensor or another type of pressure sensor. Additionally, other types of sensors, such as a temperature sensor, can also be included. The sensor 22X can be positioned within the pump housing 19X to detect an amount of air pressure in the hose 18X. For example, the sensor 22X can be positioned in a portion of the pump 14X in communication with the hose 18X, such as in fluid communication with the pressurized fluid outlet 28X of the pump 14X as shown in FIG. 1X. Since the hose 18X can be in fluid communication with the bladder 16X of the mattress 12X, the air pressure detected by the sensor 22X can indicate the air pressure in the bladder 16X. While operation of the pump 14X may affect the pressure detected by the sensor 22X, the pump 14X can operate only as required to maintain an average pressure within the bladder 16X (e.g., to replace any fluid that seeps out of the bladder 16). Additionally, the sensor 22X can draw power from a power source that also powers the pump 14X, such as the electric line 21X. The sensor 22X can output a pressure signal a to the controller 24X. The sensor 22X can be hard-wired to the controller 24X, the sensor 22X can wirelessly communication with the controller 24X by way of a transmitter using, for example, a standard wireless protocol (e.g., IEEE 802.11, RF, Bluetooth, or 3G), or the sensor 22X can otherwise be coupled to the controller 24X for communication therewith.

The controller 24X, which can be a microprocessor or another device including a memory and a CPU for executing a program stored on the memory, can control a motor 26X in the pump 14X shown in FIG. 2X to produce pressurized air in the outlet 28X portion of the pump 14X shown in FIG. 1X. The controller 24X can be hard-wired to the motor 26X or be in wireless communication with the motor 16X using, for example, a standard wireless protocol. As a result, the controller 24X can control the operation of the pump 14X. For example, the controller 24X can control the pump 14X in response to the pressure signal a such as by instructing the pump 14X to inflate the bladder 16X when the controller 24X determines the air pressure in the bladder 16X is below a set amount. Thus, when the controller 24X actuates the motor 26X, the motor 26X can produce pressurized air in the outlet 28X that passes from the pump 14X through the hose 18X and into the bladder 16X to increase the fluid pressure inside the bladder 16X. The controller 24X can also be in communication with an air release valve or other structure for releasing air from the bladder 16X such that the controller 24X can provide an instruction to decrease the fluid pressure in the bladder 16X. Also, while the controller 24X is shown as packaged with the pump 14X, the controller 24X can alternatively be packaged with the control unit 15X or some other component besides the pump 14X.

Additionally, the controller 24X can analyze the pressure signal a to determine a heart rate, respiration rate, and/or other vital signs of a user lying or sitting on the mattress 12X. More specifically, when a user lies on the mattress 12X, each of the user's heart beats, breaths, and other movements can create a force on the mattress 12X that is transmitted to the bladder 16X. As a result of the force input to the bladder 16X from the user's movement, a wave can propagate through the bladder 16X, into the hose 18X, and arrive at the pump 14X. The sensor 22X can detect the wave, and thus the pressure signal a output by the sensor 22X can indicate a heart rate, respiratory rate, or other information regarding user. If the pump 14X is of the type including a sensor 22X of the type originally designed for monitoring the fluid pressure within the bladder 16X to maintain the pressure at a substantially constant amount, a software upgrade can be used to increase the functionality of the pump 14X to determine the heart rate, respiratory rate, and other characteristics of the user without the need for a hardware modification. In this case, a hardware upgrade can provide the control unit 15X, if desired.

To overcome a DC offset in the pressure signal ?, the pressure signal ? can pass through a circuit splitting the signal into a DC coupled path and an AC coupled path, and the AC coupled path can be amplified and filtered. The controller 24X can perform a pattern recognition algorithm or other calculation based on the amplified and filtered pressure signal a to determine the user's heart rate and respiratory rate. For example, the algorithm or calculation can be based on assumptions that a heart rate portion of the signal a has a frequency in the range of 0.5-4.0 Hz and that a respiration rate portion of the signal a has a frequency in the range of the range of less than 1 Hz. The controller 24X can also be configured to determine other characteristics of a user based on the pressure signal ?, such as blood pressure, tossing and turning movements, rolling movements, limb movements, weight, the presence or lack or presence of a user, and/or the identity of the user. Further, the controller 24X can receive signals from other sensors (e.g., a temperature sensor). The controller 24X can output a status signal ? indicating the characteristics of the user (e.g., heart rate and respiratory rate) to the control unit 15X. Additionally, if multiple users are lying or sitting on the mattress 12X, the pressure signal a detected by the sensor 22X can indicate each of the multiple users' vital signs, and the pattern recognition algorithm or other calculation performed by the controller 24X can detect each user's heart rate and respiration rate.

The control unit 15X can include a transmitter 30X, a screen 32X, and controls 34X. The transmitter 30X can relay the status signal ? to a database 36X or other source. The transmitter 30X can be a wireless transmitter operating using a standard wireless protocol (e.g., IEEE 802.11, RF, Bluetooth, or 3G) for communication with the database 36X or other source, though the transmitter 30X can alternatively be hardwired to the database using a phone line, Ethernet line, or other connection. As a result, the database 36X can store sleep information produced as a result of the status signal ?, and the user can be alerted to sleep issues based on long-term sleep trends or provided with other communications regarding the user's sleep (e.g., an alarm warning of apnea), fitness level, cardiovascular condition, or other health information. An example of storing sleep information with the database is discussed below in respect to FIG. 4X.

The screen 32X can display information relayed in the status signal ?, such as a sleep score based on the user's heart rate, respiratory rate, amount of time spend in REM sleep, total time in bed, and other considerations.

The control unit 15X can also be hard-wired or in wireless communication with the controller 24X for controller operation of the pump 14X. As a result, the controls 34X can be used to control the operation of the sleep monitoring system 10X. For example, the controls 34X can be used to increase the air pressure in the bladder 16X. As another example, the controls 34X can be used to instruct the sensor 22X and/or controller 24X to operate in a privacy mode in which data is not detected, retained, displayed, transmitted, and/or analyzed, or to communicate with the database 36X to obtain sleep information (e.g., sleep trends, sleep scores from previous nights, sleeping tips). The database 36X can be accessible via the control unit 15X or a computer, e.g., via the internet.

As shown in FIG. 3X, the bladder 16X can include multiple longitudinal supports 38X spaced across the bladder 16. The supports 38X can define channels 40X for fluid in the bladder 16X to pass from, for example, the head of the bladder 16X to the hose 18X via the inlet 17X. That is, the supports 38X can be positioned not to impede waves propagating through the bladder 16X in a direction toward the sensor 24X (which in this case is through the inlet 17X). The supports 38X can also provide support for a user lying on the mattress 12X. A different arrangement of supports can be used, though the supports should not substantially hinder waves from propagating to the sensor 24X. Also, the mattress 12X can include more than one bladder 16X. For example, the mattress 12X can include two side-by-side bladders 16X for detecting the heart and respiratory rates of two users. In this case, the pump 14X can include more than one sensor 22X.

The sleep monitoring system 10X can have a different structure from illustrated. For example, the pump 14X can include the transmitter 30X instead of the control unit 15X. Additionally, the system 10X can have additional functions from those described above. For example, the control unit 15X can function as an alarm clock, and the alarm can be sounded until the system 10X determines that the user has awoken or got off the mattress 12X.

As shown in FIG. 4X, the sleep information stored in the database 36X can be used to improve the sleep of a user. In more detail, as shown in steps S1X and S2X and discussed above in greater detail, the sensor 22X can detect the pressure in the fluid bladder 16X and the controller 24X can determine at least one vital sign based on the pressure in the fluid bladder 16X.

As shown in step S3X, the database 36X can store sleep information generated over time. The sleep information can include the pressure in the bladder 16X, one or more vitals signs (e.g., heart rate, respiratory rate, etc.), a frequency or amount of tossing and turning by a user, a temperature, a light level, and other information. The sleep information need not necessarily include one of the vital signs, as one or more of the vital signs can be determined by a computer or other processing unit (i.e., a processor other than the controller 24X). Also, the sleep information can be transferred to the database 36X by communicably linking the controller 24X and transmitter 30X and also communicably linking the transmitter 30X and database 36X as shown in FIG. 2X, or in another way (e.g., directly communicably linking the sensor 22X and the database 36X, or communicably linking the sensor 22X to the transmitter 30X and the transmitter 30X to the database 36X).

The database 36X can store a log of sleep information as shown in step S3X of FIG. 4X. For example, the database 36X can create a sleep score based on one or more vital signs. The sleep score can, for example, indicate high quality sleep when heart rate is low, when respiratory rate is low, and when tossing when turning movements are infrequent. Over time, the database 36X can accumulate sleep scores for a variety of conditions (e.g., a lower pressure in the bladder 16X, a high pressure in the bladder 16X, a cool temperature, a warm temperature, and/or a low level of light).

As shown in step S4X, an association can then be made using the sleep information between the sleep score and environmental conditions, such as the pressure in the bladder 16X, the light level, and the temperature. The association can be performed by the controller 24X or another processor in communication with the database 36X. The association between the sleep score and environmental conditions can include, for example, determining a correlation between the sleep score and environmental conditions. Based on the association, a pressure setting can be determined for customizing the environmental conditions (e.g., pressure in the bladder 16X, light level, and temperature) to achieve a high sleep score. Additionally, other settings (temperature and light level, for example) can be determined based on the association.

As shown in step S5X, the controller 24X can control the pump 14X based on the pressure setting. For example, the controller 24X can actuate the motor 26X to inflate the bladder 16X if the pressure setting indicates a higher pressure would result in a higher sleep score. Further, other controls (e.g., a heater, air conditioner; and/or a night light) can be adjusted based on the association.

While the invention has been described in connection with what is presently considered to be the most practical example, it is to be understood that the invention is not to be limited to the disclosed example but, on tie contrary, is intended to cover various modifications and equivalent arrangements included within the spirit and scope of the appended examples, which scope is to be accorded the broadest interpretation so as to encompass all such modifications and equivalent structures as is permitted under the law.

### EXAMPLES

1. A sleep monitoring system comprising:

a fluid bladder;

a pump in fluid communication with the fluid bladder, the pump operable to increase a fluid pressure within the fluid bladder;

a sensor packaged with the pump, the sensor in fluid communication with the fluid bladder and operative to determine a pressure within the fluid bladder; and

a controller configured to determine at least one vital sign based on the pressure within the fluid bladder.

2. The sleep monitoring system of example 1, wherein the sensor is physically coupled to a housing containing components of the pump such that the sensor is part of an integral pump unit.

3. The sleep monitoring system of example 1, further comprising at1elongate conduit fluidly coupling the pump and the fluid bladder, and wherein the sensor is in fluid communication with the conduit.

4. The sleep monitoring system of example 1, wherein the controller is operative to control the pump in respoi1se to the pressure Within the fluid bladder.

5. The sleep monitoring system of example 1, wherein the at least one vital sign includes at least one of a hea11rate and a respiration rate.

6. The sleep monitoring system of example 1, wherein the fluid bladder encloses pathways aligned with a fluid inlet.

7. The sleep monitoring system of example 6, wherein an end of the fluid bladder defines the fluid inlet, and wherein the pathways extend longitudinally toward the fluid inlet.

8. The sleep monitoring system of example 6, wherein the fluid bladder includes parallel supports extending between a top side of the fluid bladder and a bottom side of the fluid bladder, the supports at least partially defining the pathways.

9. The sleep monitoring system of example 1, further comprising a control unit in communication with the controller, wherein the control unit includes a display configured to display. the at least one vital sign.

10. The sleep monitoring system of example 1, wherein the controller is operative to control the pressure within the fluid bladder in response to the at least one vital sign.

11. The sleep monitoring system of example 1, wherein the controller is configured to control the pressure within the fluid bladder ba5ed on an association between the pressure within the fluid bladder and the at least one vital sign.

12. The sleep monitoring system of example 11, further comprising a memory configured to store a database of sleep information generated over time including at least one of the pressure within the fluid bladder and the at least one vital sign, and wherein the association between the pressure within the fluid bladder and the at least one vital sign is determined based on the sleep information generated over time.

13. The sleep monitoring system of example 11, wherein the association between the pressure within the fluid bladder and the at least one vital sign is used to determine a pressure setting, and wherein the controller is configured to control the. pressure within the fluid bladder

to be the pressure setting.

14. The sleep monitoring system of example 1, further comprising a transmitter in communication with at least one of the sensor and the controller, and wherein the transmitter is configured-to transmit a signal corresponding to at least one of the pressure within the fluid bladder and the at least one vital sign to a memory.

15. The sleep monitoring system of example 1, wherein the system is operable in a privacy mode in which at least one of sensing the pressure within the fluid bladder, determining the at least one vital sign, recoding the at least one vital sign, outputting the at least one vital sign, and displaying the at least one vital sign is turned off.

16. A sleep monitoring system comprising:

a fluid bladder;

a pump spaced from the fluid bladder, the pump having a housing containing pump components and defining a fluid inlet for receiving fluid and a fluid outlet for outputting fluid pressurized by the pump;

an elongate conduit fluidly coupling the fluid outlet of the pump and the fluid bladder, the conduit providing a passage for the fluid pressurized by the pump to increase a fluid pressure within the fluid bladder;

a pressure sensor disposed within the interior of the pump housing such that the sensor is part of an integral pump unit, the pressure sensor configured to detect a pressure of fluid at the fluid outlet of the pump; and

a controller coupled to the sensor and configured to determine at least one vital sign based on the pressure within the fluid bladder.

17. The sleep monitoring system of example 16, wherein the controller is configured to control the pressure within the fluid bladder based on the at least one vital sign.

18. The sleep monitoring system of example 16, wherein the controller is configured to control the pressure within the fluid bladder based on a user-specific database of sleep information generated over time.

19. A sleeping pad comprising:

a fluid bladder;

a pressure sensor in fluid communication with the fluid bladder and operable to detect a fluid pressure within the fluid bladder;

a controller in communication with the pressure sensor and operable to determine at least one vital sign of a user based on the pressure within the fluid bladder;

a memory for storing historical data including the pressure within the fluid bladder and the at least one vital sign;

a processor for determining a sleep quality correlation between the pressure. within the fluid bladder and the at least one vital sign based on the historical data; and

a pressurized fluid source operable to increase a pressure within the fluid bladder when the sleep quality correlation indicates a sleep quality of the user would improve if the pressure within the fluid bladder were higher.

20. The sleeping pad of example 19, wherein the fluid bladder includes a fluid outlet. operable to release fluid from the fluid bladder when the sleep quality correlation indicates a

sleep quality of the user would improve if the pressure within the fluid bladder were lower.

This application claims the benefit of priority to U.S. Provisional Application No. 60/846,642, filed Sep. 22, 2006, which is hereby incorporated in its entirety by reference.

The present method and apparatus relate to monitoring vital signs, such as the presence of a heartbeat and breathing, in an human or animal, e.g., an infant sleeping in a crib, a patient in a hospital setting, a person with a chronic disease, a person in an elder-care setting, or an animal at home or in the care of a professional.

There are a variety of settings in which monitoring one or more vital signs of an individual can be important. For example, sleeping infants may be monitored for respiration or heartbeat, to alert parents or guardians to changes in the infant's heart of breathing status, caused for example, by sudden infant death syndrome (SIDS) or accidental suffocation. In a hospital setting, such as an emergency room (ER) waiting area, ambulance, or where an individual has been hospitalized with a cardiac condition, it may be important to monitor the individual's heart rate, both to provide an alert for a catastrophic systems failure or to monitor changes in heart rate status, e.g., in response to certain medications. Elder care patients who are at risk for cardiovascular failure or decline may also need vital-signs monitoring, to alert an attendant to dramatic changes in health status or the need for drug intervention. Typically, these are all settings in which an individual is lying or sitting at rest.

A number of devices designed for monitoring vital signs are known. In a hospital setting where a patient's heart rate and function are being monitored, it is usual to record a continuous electrocardiogram (EKG or ECG) of the patient. This is performed by attaching a number of electrodes to various points of the patient's chest and back region, to measure the rhythmic electrical activity of the heart. An EKG hookup tends to be uncomfortable and confining over extended periods, and requires a trained medical professional to apply the electrodes properly and to operate the device. For example, during sleep, the electrodes can come off the patient and cause a false alarm. The cost and inconvenience of EKG monitoring make it impractical for many health-monitoring settings, such as non critical hospital patients, infant or elder care monitoring.

A less intrusive means for measuring heart rate is a mechanical inertial device known as a ballistocardiograph, which is designed to record the volume of blood passing through the heart, at any period in time, based on the body's recoil as blood is ejected from the heart ventricles with each heartbeat. Such devices, as exemplified by U.S. Pat. No. 4,679,569, tend to have a rigid, mechanical plate structure and a complicated mechanism for measuring changes in plate motion.

There exists a need for a monitoring apparatus that has a simplified, relatively inexpensive construction, can be used comfortably by an individual on a mattress or chair, does not require any patient hookup, can be used easily by an untrained person, and provides accurate heart and respiration-rate information to a monitoring site or person.

The invention includes, in one aspect, an apparatus for monitoring heart and respiration rates of a human subject at rest, comprising, in operative condition,

a sensing unit having (i) a fluid or gas-filled pad adapted to be placed on a bed, crib, or chair support, for cushioning at least an upper body portion of a subject lying on or resting against the support, mattress or cushion, and (ii) a pressure sensor in fluid communication with gas or fluid in said unit, for generating electrical signals in response to pressure variations within the gas or fluid in the pad, and

a monitoring unit operatively connected to said pressure sensor, for (i) receiving signals therefrom, (ii) generating from said signals, information about the heart and respiration rates of the subject, and (iii) relaying such information to a monitoring station or individual.

In some embodiments, the pad is a fluid-filled pad. In some embodiments, the pad is a gas-filled pad.

In some embodiments, the apparatus further includes a pressure-control unit comprising a pump in fluid or gas communication with the pad and a controller operatively connected to the pump for maintaining fluid or gas within the pad at a selected pressure or within a defined pressure range.

In some embodiments, the pad comprises a single fluid or gas-filled chamber, having a pressure sensor in fluid or gas communication therewith, for generating electrical signals in response to pressure changes within the chamber.

In other embodiments, the pad includes at least two independent, fluid or gas-filled chambers, each of which has a pressure sensor in fluid communication therewith, for generating electrical signals in response to pressure changes within the associated chamber.

In some embodiments, the apparatus further includes an ambient-null device comprising a fluid or gas-filled ambient pad, a weight carried on the ambient pad, for exerting pressure thereon, and an ambient pressure sensor in fluid or gas communication with fluid in the ambient pad, for generating electrical signals in response to pressure changes within the fluid or gas, in response to ambient motion in the vicinity of the subject, wherein said monitoring unit is operatively connected to said ambient pressure sensor, for (i) receiving signals therefrom, and (ii) processing the signals received from the first-mentioned and ambient pressure sensors, to filter such ambient motion from motion related to the subject's heart and respiration rates.

In some embodiments, the monitoring unit includes a processor operative to (i) generate heart-rate information of the subject, based on time-dependent signals having frequency components in the range from about 0.1 to about 10 Hz, and (ii) generate respiration rate information of the subject based on time-dependent signals having frequency components in the range less than about 1 Hz. In particular embodiments, the information generated by the signal processor further includes blood-pressure information.

In some embodiments, the pad includes upper and lower independent, fluid or gas-filled chambers, each of which has a pressure sensor in fluid communication therewith, for generating electrical signals in response to pressure changes within the associated chamber, and the information generated by the processor includes information about the orientation of the individual with respect to the pad, based on a characteristic ventral, dorsal or lateral signals produced by processing the two separate signals generated for the two chambers.

In some embodiments, the monitoring unit includes a remote monitor, and a transmitter for transmitting such heart and respiration rate information from the processor to the monitor.

In some embodiments, the pad further includes temperature sensor for measuring the temperature of the individual on the pad.

In another aspect, a sensor unit for use with a monitoring unit is provided, for monitoring heart and respiration rates of a human subject at rest, comprising, in operative condition,

a fluid or gas-filled pad adapted to be placed on a bed, crib, or chair support, for cushioning at least an upper-body portion of a subject lying on or resting against the support, and

a pressure sensor in fluid communication with fluid in said unit, for generating electrical signals in response to pressure variations within the fluid or gas, and adapted to be operatively connected to such a monitor.

In some embodiments, the pad includes as single fluid or gas-filled chamber having a pressure sensor in fluid of gas communication therewith, for generating electrical signals in response to pressure changes within the chamber.

In other embodiments, the pad includes at least two independent, fluid-filled chambers, each of which has a pressure sensor in fluid or gas communication therewith, for generating electrical signals in response to pressure changes within the associated chamber. In some embodiments, the pad is a fluid-filled pad. In some embodiments, the pad is a gas-filled pad.

In another aspect, a method for monitoring vital signs is provided, including heart and respiration rates, of a human subject lying on or resting against a bed, crib, or chair support, comprising

placing between the subject and the support, a fluid or gas-filled pad positioned for cushioning at least an upper-body area of the subject,

generating electrical signals in response to pressure variations within the fluid or gas by a pressure sensor in fluid communication with fluid or gas in said pad, and

processing the electrical signals received from the pressure sensor to generate information about the heart and respiration rate of the subject.

In another aspect, an apparatus for remotely monitoring heart and respiration rates of a human subject lying on or resting against a bed, crib, or chair support is provided, comprising

a pad adapted to the placed between the subject and the support, for cushioning at least an upper body portion of the individual,

a sensor on said pad for generating motion-related signals caused by the subject's heartbeat and breathing,

a processor operatively connected to said sensor, for (i) receiving time-dependent signals therefrom, and (ii) generating heart-rate information of the subject, based on received time-dependent signals in the range from about 0.1 to about 10 Hz, and respiration rate information of the subject, based on received timed-dependent signals in the range less than about 1 Hz,

a remote monitor for use by an individual in monitoring said subject, and

a transmitter for transmitting such subject information from the processor to the individual.

In some embodiments, the apparatus further includes an ambient-motion device for generating signals related to ambient motion in the vicinity of the subject, and said processor is operatively connected to said device, for processing the signals received from the device, to filter such ambient motion from motion related to the subject's heart and respiration rates.

In another aspect, an apparatus for monitoring vital signs is provided, including heart and respiration rates, of a human subject lying on or resting against a bed, crib, or chair support, comprising

a pad adapted to the placed between the subject and the support, for cushioning at least an upper body portion of the individual, said pad comprising

a pair of confronting plates, one adapted to be supported on the mattress, and the other adapted for contact with the chest area of the individual, said plates being spaced apart for relative lateral movement in an XY plane and relative vertical movement in the Z direction,

(iii) connecting the two plates, an L-shaped connector attached at opposite ends to the opposing plates and having a pair of laterally extending, orthogonally disposed arms, a strain gauge carried on each arm, in an XY plane, and a strain gauge carried on one of the arms, in a vertical plane, and

(b) a monitoring unit operative to transmit to a remote user, information about the heart rate of the individual, based on signals received from the pad's lateral-movement strain gauge devices, and about the respiration rate of the individual, based on signals received from the pad's vertical-movement strain gauge(s).

The apparatus of example 20, wherein said two opposing plates are substantially rectangular, and connected by said L-shaped connectors in the region of each of the four corners of the two plates.

In some embodiments, the apparatus further includes a vertical-movement strain gauge connecting the two plates, for generating information about the weight applied by the individual on the pad.

In some embodiments, the monitoring unit includes a processor operative to (i) wherein said monitoring unit includes a signal processor operative to (i) generate heart-rate information of the subject, based on time-dependent signals received from each of the plural lateral-movement strain-gauge devices, and having frequency components in the range from about 0.1-10 Hz, and (ii) generate respiration rate information of the subject based on timed-dependent signals having frequency received from the at least one of the vertical-movement strain gauge(s), and having frequency components in the range less than about 1 Hz.

In some embodiments, the monitoring unit includes a remote monitor, and a transmitter for transmitting such heart rate and respiration rate information from the processor to the monitor.

In some embodiments, the pad further includes temperature sensor for measuring the temperature of the individual on the pad.

In some embodiments, the apparatus further includes a weighted strain gauge adapted for attachment to the bed or crib, independent of said pad, for detecting movement of the bed or crib, independent of movement within the pad, and the monitoring unit is operative to remove such independent movement from pad movement detected by the pad strain gauges.

In a related aspect, an apparatus for determining the presence of a subject is provided, comprising:

a sensing unit having (i) a fluid or gas-filled pad adapted to be placed on a bed, crib, or chair support, for cushioning at least an upper body portion of a subject lying on or resting against the support, mattress or cushion, and (ii) a pressure sensor in fluid communication with fluid in said unit, for generating electrical signals in response to pressure variations within the fluid in the pad, and

a monitoring unit operatively connected to said pressure sensor, for (i) receiving signals therefrom, (ii) generating from said signals, information about the presence of the subject and (iii) relaying such information to a monitoring station or individual.

In another related aspect, a sensor unit for use with a monitoring unit, for detecting the presence of a subject is provided, comprising:

a fluid or gas-filled pad adapted to be placed on a bed, crib, or chair support, for cushioning at least an upper-body portion of a subject lying on or resting against the support, and

a pressure sensor in fluid communication with fluid in said unit, for generating electrical signals in response to pressure variations within the fluid or gas, and adapted to be operatively connected to such a monitor.

A related method for detecting the presence of a subject on or in a bed, crib, or chair support is provided, comprising:

placing on or in the bed, crib, or chair support a fluid or gas-filled pad positioned for cushioning at least an upper-body area of the subject,

generating electrical signals in response to pressure variations within the fluid or gas by a pressure sensor in fluid communication with fluid or gas in said pad, and

processing the electrical signals received from the pressure sensor to generate information about the presence of the subject.

In a related aspect, an apparatus for monitoring the presence of a subject lying on or resting against a bed, crib, or chair support is provided, comprising:

a pad adapted to the placed between the subject and the support, for cushioning at least an upper body portion of the individual, said pad comprising

a pair of confronting plates, one adapted to be supported on the mattress, and the other adapted for contact with the chest area of the individual, said plates being spaced apart for relative lateral movement in an XY plane and relative vertical movement in the Z direction,

(iii) connecting the two plates, an L-shaped connector attached at opposite ends to the opposing plates and having a pair of laterally extending, orthogonally disposed arms, a strain gauge carried on each arm, in an XY plane, and a strain gauge carried on one of the arms, in a vertical plane, and

(b) a monitoring unit operative to transmit to a remote user, information about the presence of the subject, based on signals received from the pad's strain gauge devices.

Apparatus for monitoring the presence of a subject, rather than health of a subject, may be connected to the internet and may further include any of the additional features described herein.

These and other aspects and embodiments of the present invention will become better apparent in view of the detailed description in conjunction with the accompanying drawings.

### I. INTRODUCTION

A method and apparatus are provided for monitoring the presence and health status of human and animal subjects/patients. The method and apparatus utilize a pad or plate sensor unit adapted to be placed in a bed, cushion mattress, infant crib, or the like for generating health status data corresponding to the subject's cardiac function and/or respiration (i.e. breathing). The pad or plate sensor may be a fluid or gas-filled device, an electromechanical device, an optical device, or a semi-conducting device, depending on the embodiment.

Data generated from the sensor unit may be combined with additional data (e.g., generated by one or more additional sensors), filtered, and relayed to a microprocessor for recording or analysis. Processed data may be used to trigger one or more events. In some examples, the event is to sound an alarm or alert medical professionals to deteriorating health status of a subject. The health status data that trigger an event, and the events that are triggered, may be pre-selected by a user depend on the particular application.

The method and apparatus are readily integrated with internet/web-based services, wireless telecommunications, advanced audio and video processing, instant messaging, digital and analog signal processing, medical record databases and patient records, and private and public health agencies, thereby linking a subject's health status to any number of services.

FIG. 1Y shows an exemplary monitoring method and apparatus. The view illustrates an infant crib 6Y with a sensor pad or plate sensor 1Y adapted for use as a mattress. A wireless in home monitor 2Y is provided, e.g., to allow a parent or guardian 7Y to monitor data from the sensor 1Y via a wireless phone or internet protocol link 3Y. The crib is further equipped with a camera 4Y to transmit live or delayed video, e.g., to allow the determination of whether the infant, adult, or animal is on its back, front, or sides, by identifying features of the subject, and a panic button 5Y. A wired or wireless transceiver can also be equipped to communicate between the sensor and the camera, panic button and remote monitor. As shown in FIG. 2Y, the pad or plate sensor 1Y detects heart 9Y and respiration vibrations in from the infant subject 10Y along with data from optional additional sensors (i.e., a microphone 7Y and thermometer 8Y). These data are transmitted, by wire or wirelessly, to a digital signal processor (DSP) 11Y, which analyzes the data and triggers appropriate actions.

The method and apparatus are described in more detail, below.

### II. MONITORING APPARATUS

A feature of the present method and apparatus is a sensor unit adapted for placement on a bed, crib, chair, automotive or avionics seat, or similar rest surface for a human or animal. In some embodiments, the sensor is in the form of a mattress or mattress pad, upon which a subject will rest. In other embodiments, the sensor is in the form of a cushion or cushion pad, upon which a subject will sit or lean. In other embodiments, the sensor is in the form of plate upon which a subject will rest.

Both fluid/gas-filled sensors and electromechanical sensors may be used according to the present method and apparatus. Such sensors may be referred to as ballistocardiographs, monitor-enabled pads or mattress, vital signs sensors, or health status data sensors.

Embodiments of the pad or plate sensor are described, below.

Fluid/Gas-Filled Pad Embodiment

In some embodiments, the sensor uses a fluid or gas-filled pad upon which a subject will rest. The fluid/gas-filled pad may be connected to a suitable fluid/gas pump to maintain a desirable pressure and/or volume in the pad. The pad is further connected to an fluid/gas pressure sensor, which monitors the pressure changes in the pad in response to a subject's hear function or respiration. According to this embodiment, incident pressure waves caused by shifting body weight in response to cardiopulmonary activity induces a change in the measured pressure, which data are sampled and processed. This embodiment of the method and apparatus are illustrated in FIGS. 3Y-5Y.

As shown in FIG. 3Y, a custom air mattress 10Y is operably connected to an air pump 21Y for filing the pad sensor to a preselected pressure or volume and an air pressure sensor 31Y for monitoring the pressure in the mattress 10Y. Ballistic motion of the subject infant 100Y caused by cardiac function and breathing cause pressure variations in the pad sensor 10Y, which can be detected by the pressure sensor 31Y, which produces or alters electrical signals in response to pressure variations. A signal (i.e., data; typically electrical) from the pressure sensor 31Y is received by a microprocessor 200Y for analysis. The raw or processed signal/data may be sent to the internet 300Y for distribution.

FIGS. 4YA-YD illustrate several embodiments of an air or fluid-filled pad sensor 10Y, shown from the side (beneath an infant 100Y) and from the top. FIG. 4YA illustrates a single chamber pad sensor. FIGS. 4YA-4YC show different configurations of multiple chamber pad sensors, where lines or a grid indicate the separate chambers. Each chamber may be connected to a separate pressure sensor or multiple chambers may be connected to a single pressure sensor (not shown). The dark ovals in each panel represent conventional structures within the mattress. The pad may include any number of ribs, which may be part of the individual chambers. In some embodiments, the pad includes a single chamber. In other embodiments, the pad includes at least two chambers. In related embodiments, the pad includes a plurality of chambers. Where the pad sensor includes a plurality of chambers, the chambers may be vertically or horizontally stacked. The subject may rest on a stack of chambers or may rest on several adjacent chambers.

FIG. 5Y illustrates and embodiment that employs an air or fluid-filled pad 10Y for monitoring a subject's 100Y cardiac and/or respiratory function and an ambient null sensor device 50Y for monitoring ambient motion in the vicinity of the subject 100Y. The air or fluid-filled pad 10Y and ambient null device 50Y are separately connected to pressure sensors 30Y, 31Y, which provide pressure data for filtering and analysis by a microprocessor 200Y. The air or fluid pump 20Y for filing the pad sensor 10Y is indicated. The same or a different pump 20Y may be connected to the ambient null device 50Y (not shown).

Where an ambient and null device/sensor is used in combination with a pad sensor, the signal from the null device may be subtracted from (i.e., used to “null” or “cancel out”) the signal from the pad sensor, allowing background signal (i.e., noise) subtraction.

In some embodiments, the pad sensor is filed with air. In related embodiments, the pad sensor is filled with an inert gas. In other embodiments, the pad is filled with a fluid. In particular embodiments, the fluid is an aqueous solution or water, optionally with an additive to retard the growth of microorganisms. Preferred fluids are inexpensive and non-toxic. Air-fluid emulsions or hybrid air/fluid configurations should produce similar results.

Pad sensors may be made of virtually any conventional material that is air or water-tight, as required by the particular embodiment. Exemplary materials include but are not limited to plastic (e.g., polyethylene, polypropylene, latex, vinyl, etc.) and fabric (e.g., canvas). Fabrics may be treated with a plastic or other coating to make them air or fluid-tight, as required. The pad may be covered for comfort or protection, so long as the covering does not substantially insulate the sensor from the vibrations generated by the subjects heart and/or lung function.

Where the pad sensor includes multiple chambers (e.g., FIGS. 4YB-4YD), each chamber may be operably connected to a separate pressure sensor or a plurality of chambers may be connected to a single pressure sensor. Generally, one pad is used for each subject. Where a single pad is used to monitor a plurality of subjects, e.g., as in the case of a large pad for monitoring a plurality of infants or adults, a plurality of chambers is preferred, thereby allowing distinction between the heart and respiratory functions of each subject on the mattress sensor.

The pad may include foam or ribbing to provide structural support, to reduce resonance or harmonics, or to preventing “bottoming out” under the weight of the subject. Foam may also allow for self-inflating of the pad. Ribbing may be the result of compartmentalization or chambers, as described above. Ribbing may also be used to focus the incident waves on the pressure sensor. In some embodiments, the pad sensor is in the form of a “U” shape to force incident waves to the ends of the tube, where the pressure sensor is typically located.

While changes in the dynamic pressure in the pad are used to monitor cardiopulmonary health status data (i.e., vital signs), static pressure in the pad sensor can be used to measure a subject's weight. In this manner, the pad sensor can also be used to provide weight data (e.g., over time), or to detect the presence or absence of the subject on the pad.

### B. Mechanical Plate Embodiment

In some embodiments, the cardiac and respiratory functions are monitored using a mechanical plate (or electromechanical) sensor. In a particular embodiment, the plate sensor includes at least one weighted strain gauge for detecting vibrations resulting from cardiac and/or respiratory functions of a subject.

An embodiment of the method and apparatus that employs a strain gauge is shown in FIGS. 8YA and 8YB. As shown in FIG. 8YA, the plate sensor apparatus comprises an upper plate 60Y and lower plate 61Y. The subject 100Y rests on the upper plate 60Y. As shown in FIG. 8YB, the upper and lower plates are connected via one or more strain gauges 71Y, 72Y, 7Y, 74Y, each having a first end 62Y attached to the upper plate 60Y and a second end 63Y attached to the lower plate 61Y. The strain gauges may be adapted to measure strain in any dimension, such as the X, Y, and Z, axes as shown in FIG. 8BY. Strain gauges may also measure the rotation of one plate with respect to the other, the tilting of one plate with respect to the other, or the flexing of the upper or lower plate.

Ballistic movement of the subject in response to heart and lung function is generally not limited to a single direction. In some embodiments, it may be desirable to monitor movement in several directions to increase the sensitivity of the plate sensor. However, it is generally not necessary to monitor movement in all directions. In some embodiments, it may be adequate to monitor movement in one direction. Thus a limited small number of strain gauges, such as 1, 2, 3, 4, 5, or 6 should be sufficient to detect cardiac and/or lung function. The two plates may further be connected by springs, foam, an air or fluid-filled bag or cushion, etc. to maintain a nominal separation distance between the plates. The weight of the intended subject will be reflected in the springs, foam, or other material used to maintain distance between the plates.

FIG. 9Y is a schematic diagram showing how an electromechanical sensor is used according to the method and apparatus. Electrical signals from strain gauges or pressure sensors measuring movement in the X 81Y, Y 82Y, and Z 83Y axes, along with (optionally) electrical signals from other sensors, such as a microphone 84Y and temperature gauge 85Y are fed into filters 90Y, received by an analog to digital converter 95Y, or similar device, and analyzed by a digital signal processor (DSP) 200Y. The DSP includes preselected or learned/trained parameter information (arrows pointing down towards DSP 200Y) and may trigger one or more events (arrows point away from DSP 200Y). The DSP 200Y may also communicate with a wireless transceiver 400Y for further distributing the processed signal.

### C. Further Embodiments

Combinations of gas/fluid pressure sensors and strain gauges may be used to increase the sensitivity of detection of vibrations resulting from heart and lung function. In addition, other types of sensors may be used in addition to, or in place of, gas/fluid-filled and electromechanical sensors. Cost and practicality should be considered in the design. The above-described sensors offer adequate sensitivity without being overly elaborate in design.

Although preferred health status sensors are non-invasive, non-entangling, and unobtrusive, some embodiments employ a sensor that is worn or attached to the subject, e.g., in the form of a wrist or ankle-worn sensor. Such sensors may be adapted to communicate with a processing or analytical device in a wireless manner, thereby minimizing the intrusive nature of the sensor.

### III. ADDITIONAL SENSORS

In addition to the pad or plate sensor for detecting vibrations from heart function and/or breathing, the method and apparatus may include one or more additional sensors for obtaining health status or environmental data. Such additional sensors include but are not limited to temperature sensors for monitoring ambient temperature and/or the temperature of the subject; light sensors for monitoring ambient light; weight sensors for measuring subject weight, moisture sensors for detecting bed-wetting or other nocturnal emissions; audio and/or video sensors for detecting crying, fussing/complaining, snoring, tossing and turning, position indicators for detecting changes in mattress angle, changes in the subjects orientation, etc.

Exemplary additional sensors include microphones, cameras, thermometers, photoelectric devices, microelectromechanical sensors (MEMS), sphygmomanometers, strain gauges, accelerometers, inclinometers, altimeters, barometers, radiation detectors, moisture gauges, and the like. In some embodiments, the additional sensors obtain data in a non-invasive manner, much like the pad sensor. In other embodiments, the additional sensors are connected to the subject. Data from such additional sensors can be used passively, i.e. recorded for later use; sent periodically to web pages or cell phones; displayed on a monitor, etc. Data from such devices can also be used actively, i.e. used to determine ambient light, detect motion via frame differencing, triggering an alarm, etc. Exemplary additional sensors are exemplified, below:

Ambient Light Monitors

Ambient light monitors (photo detectors, photo diodes, CCD integrators, etc.) can be used to capture and track the amount of light in the room occupied by the subject. By looking at the spectral components, it is also possible to determine if the source is natural or artificial light.

### B. Video Capture Device

Video capture devices, such as visible-light or infrared (IR) cameras, can be used to take snapshots, time lapse images, or continual frames of the subject. In some embodiments, data from a video capture device is used to trigger a wake-up alarm, turn on or off lights, etc. Data from an infrared detector may be used to monitor the temperature of a subject. Video data may also be used to determine the position of a person or animal, as well as when the person/animal has turned over.

### C. Audio Sensors

Audio sensors, such as microphones, can be used to identify crying, coughing, snoring, screaming, hiccoughing, groaning, and/or “fussiness.” Microphones are well known in the art.

### D. Temperature Sensors

Temperature/thermal/IR sensors can be used to monitor ambient room temperature and/or a subject's body temperature. Where the temperature sensor measure a subject's temperature, it may be placed on the top of the mattress sensor or built into the pad or plate sensor. Non-contact thermometers are particularly useful for measuring a subject's body temperature.

### E. Chemical Sensors

Chemical sensors can be used for warning and/or diagnosis. For example, carbon monoxide, carbon dioxide, oxygen, natural gas, methane, hydrogen sulfide, and ammonia sensors can be used to identify life threatening environmental conditions caused by, e.g., poor ventilation, smoke, fire, etc. Chemical sensors may also be used to monitor flatulence or metabolic conditions that result in the production of detectable chemical species (e.g., ketosis, trimethylaminuria). A carbon dioxide sensor may be utilized to determine if an infant has rolled over onto its front, a potential condition for suffocation. A vast number of chemical sensors are available, depending on the chemicals likely to be present in the particular environment.

### F. Weight Sensors

In some embodiments, it may be desirable to monitor a subject's body weight in addition to the subject's cardiac and/or respiratory function. Body weight monitoring is readily accomplished using a conventional scale, which is typically placed under the mattress sensor.

Body weight may also be determined from the average (i.e., static as opposed to dynamic) pressure in the pad sensor or on the plate sensor, which corresponds to the weight of the subject. In this manner, the pad or plate sensor may serve as both a cardiac function/breathing monitor and a weight sensor (or scale).

### G. EKG/EEG

Electrocardiographs (EKG; ECG) may be used to supplement data from the pad sensor, to calibrate the pad sensor, or to detect particular cardiac abnormalities.

In some embodiments, electroencephalograph (EEG) data is obtained from a subject to monitor brainwaves. This embodiment is particularly useful for studying sleep patterns in subjects and for monitoring subjects for brain activity following a stroke, heart attach, or trauma.

### H. Movement Sensors

In some embodiments, movement (or motion) sensors are used in combination with the pad or plate sensor to detect the presence of the subject in the room, to determine whether a crib, bed, chair, sofa, etc. is occupied, to monitor gross subject movements. Movement sensors include inclinometers, accelerometers, photodetectors, and the like.

### IV. AMBIENT NULL SENSOR DEVICE

In some embodiments, the pad or plate sensor is used in combination with an ambient (or null) sensor device for measuring ambient motion in the vicinity of the subject. In preferred embodiments, the ambient null device is similar to the pad or plate sensor for monitoring cardiopulmonary vibrations, differing in that the subject does not rest on the ambient null sensor. In other embodiments, the ambient null device is a device different from the pad or plate sensor, including but not limited to an accelerometer or bob weight device.

The ambient null device is used as a “control” for environmental changes that are not due to movement of the subject in question. The signal/data from the ambient null sensor can be subtracted from that of the pad or plate sensor to reduce background noise and account for changes in the environment in which the subject is resting on the pad or plate sensor.

In preferred embodiments, the ambient null device incorporates a sensor similar to that of the pad or plate sensor, such that the data produced are comparable. In some embodiments, the sensor is of the same type or model. Alternatively, the ambient null sensor is of a different type that the pad or plate sensor, including any of the sensor types described herein.

Not all embodiments of the present method and apparatus require use of an ambient null device/sensor. Vibrations resulting from heart function and breathing are regular and rhythmic and not easily confused with ambient noise; therefore, it should generally not be necessary to use an ambient sensor device unless suitable analog or digital filters, including software filters, cannot be designed. Ambient null devices are generally only required where background noise (including noise from other human or animal subjects) interferes with detection and monitoring of cardiac function and/or respiration.

### V. DATA PROCESSING

Raw data from a pad or plate sensing unit and, optionally, other sensor(s) and inputs, are processed to produce processed data. Processing may be by analog means or by digital means.

FIG. 6Y shows a typical data processing arrangement. Input data from, e.g., one or more pressure sensors or strain gauges 61Y and optional additional sensors 62Y are filtered using band-pass filters 63Y, 64Y, 65Y, amplified, and digitized, e.g., using an analog to digital converter 66Y. The filtered signals are then sent to a DSP 67Y for further processing and/or analysis. The DSP 67Y may trigger alerts, alarms, or events directly and/or may be sent to a remote location using a wireless transceiver 68Y. The remote location may be, e.g., the internet or a remote monitor. In other embodiments, input data is first digitized and then filtered or otherwise processed. Data from different sensors may be processed differently.

FIG. 7Y shows exemplary cardiopulmonary data obtained from an infant placed on a pad sensing unit as described. The raw sensor data were processed through a 10 Hz low-pass filter, amplified, digitized, digitally band-passed, and then fed to a fast Fourier transformer to convert the data to the frequency domain. Similar results could have been obtained by amplifying and digitizing the raw signal and using a digital/software low-pass filter. Since the beating frequency of a human heart is approximately 50-200 beats per minute (0.83-3.33 Hz) the frequency range of interest for monitoring human (and many other animal) heart rates is from about 0.1 to about 10 Hz, or from about 1 to about 5 Hz, or even from about 2 to about 5 Hz. Since the respiration/breathing frequency of a human is about 10-20 breaths per minute (0.16-0.33 Hz) the frequency range of interest for monitoring human (and many other animal) breathing rates is from about 0.1 to about 1 Hz but generally less than about 1 Hz.

Analog and/or digital filters can be used to select any portion of a signal for analysis. Other frequency ranges may be of interest, e.g., for monitoring coughing, screaming, hiccoughing, snoring, groaning, turning, flipping, shivering, shaking, convulsions, movements in dreams, erotic stimulation, or other movement.

Processed data can be analyzed by a microprocessor and used to trigger an event or event set, such as alerting medical professionals to assist in identifying, preventing, or treating the subject, sounding an alarm, etc, as described. The event set that is triggered depends on the rules created or tailored by the user. Examples include sending a message via the internet, logging an entry in a log file, changing a database entry, and the like. Data can also be recorded, with our without accompanying analysis, for later reviewed.

The present method and apparatus are ideally integrated with internet/web-based services, wireless telecommunications, advanced audio and video processing, instant messaging, digital and analog signal processing, medical record databases and patient records, and private and public health agencies.

Where the method and apparatus are connected to the internet, filters and/or microprocessors used to process raw data and/or analyze processed data may be at a location remote from the sensing unit. In one embodiment, raw data are transmitted via an internet connection to a microprocessor associated with a server. In another embodiment, data processed by a local microprocessor are transmitted via an internet connection to a microprocessor associated with a server.

### VI. EXTERNAL DEVICES AND PLATFORMS

In some embodiments, it may be desirable to use in the present method and apparatus in combination with an external device or platform, such as a text messaging platform, data logger, printer, alarm system, alert siren, or other data acquisition or actuating device; or a computer (i.e., microprocessor) capable of performing analytical functions.

In some embodiments a message platform is used for delivery of data, messages, alarms, and alerts. These messages may take, for example, the form of text messages (short message service, SMS) sent by way of telephone services, email, voice calls, and in home monitoring media including audio, video, and heart and breathing sounds, either in the form of direct audio, or simulated sound processes. Telephone services utilized by embodiments of the invention may include either or both the public switch telephone network (PSTN) connections and cellular telephone connections as well as a IP network connection.

Alarms or alerts may be triggered by processed signal data that are outside normal values or meet pre-selected user trigger points. Such alarms or alerts may be delivered by a telephone, web, or other service, as described. Alarms or alerts may be sent to e.g., pre-selected health care professionals (including paramedics, physicians, nurses, police, and the like), relatives and/or guardians, public health agencies, child services, etc., as determined by the user. Simple alarms or alerts are audible and/or visible signals, such as horns, buzzers, sirens, lights, and the like.

Alarms, alerts, and/or panic signals may also be localized to particular places in a home, hospital, elderly, care facility, or infant care facility. Such signals may transmitted by wired or wireless technology, such as cabling, WiFi, Zigbee, Bluetooth, etc., for contacting receiving devices such as cell phones or personal digital assistants (PDAs).

Some embodiments may also include a “panic button” that can be manually activated by the subject or another person. The panic button may cause a signal to be sent to pre-selected health care professionals, relatives and/or guardians, public health agencies, child services, etc., as above. As above, the signal can be sent via a telephone, the web, or another service, as described.

In some cases, it may be desirable to trigger an automatic action in response to processed data. For example, it may be desirable to disturb a subject's sleep with an audible and/or visible signal or through vibration, shaking, or physical contact with the subject. In other embodiments, pre-selected health status data causes, e.g., medication to be dispensed to a patient, a respirator to begin pumping air, a defibrillator to restart a subject's heart, a portion of a mattress to be raised or lowered, etc.

In some embodiments, the external device is a data logger or recording device for keep track of a subject's health status data. In other embodiments, a printer of chart recorder is connected. Most any of the described external devices can be used in combination.

FIG. 10Y shows an exemplary system in which data from a pad or plate sensor (and optional additional sensors) is communicated to a microcontroller 92Y via a wireless transceiver 91Y. The microcontroller 92Y analyzes the data, which may be viewed or presented on a remote monitoring device 93Y, in addition to being sent to the internet, being used to trigger event sets, etc. The remote monitoring device could be located, for example, in a physician's office, a nurse's station, a fire department or paramedic station, a parent's or guardian's bedroom, etc.

In all cases, the method and apparatus make include two-way (or more) communication between subject and a remote monitoring location. The two-way communication may be audio, e.g., using microphones and speakers; video, e.g., using cameras and monitors; or text, e.g., using email, messaging, or the like.

### VII. INTERNET CONNECTIVITY

Embodiments of the method and apparatus include a web portal, as part of the monitoring capability. The web portal is supported by a web server through which users may access the web. Connection to a web portal also provides access to a back-end server to capture, store, and analyze data from the various sensors of the system. The web portal typically includes an interface for the user to set various pre-selected parameters, such as which data triggering alerts or alarms.

In some embodiments, the interface provides access to a user's account (typically the subject's account), where preferences are pre-selected, and where billing and management are handled. The interface may further provide storage, presentation, and delivery of data that have been recorded. The data may be annotated with, for example summaries and analyses. The web portal may further provide drug recommendations, advertising material, news, tips, or other information based on health status data collected from the subject.

Connectivity to the internet and/or local area networks permits the pad or plate (or additional) sensors of the present method and devices to be linked to patient/invalid monitoring devices, alert services, and web applications for transmitting, receiving, and storing health data. In particular embodiments, the method and device are used to provide alerts or alarms in response to an adverse cardiovascular or respiratory event. Alerts generated by the system may be directed to health care professionals, family members, to a data logging device, or to emergency service agencies such at police, fire, ambulance, medic, etc.

In some embodiments, a web-based service specifically designed to monitor a plurality of subject using separate pad or plate sensors, is provided. The subjects may be in different locations. The web service may analyze data and determine a course of action, which can include any of the alerts, alarms, or events described.

### VIII. PATIENT POPULATIONS AND SETTINGS

The invention provides a method and apparatus for the non-invasive, non-entangling, and unobtrusive health status monitoring of a subject in a home or health care institutional setting, particularly with respect cardiovascular health status. A healthcare institutional setting may be a physicians's office, hospital, clinic, nursing facility, veterinary clinic, or assisted living facility, by way of examples.

The method and apparatus may be used to monitor “vital signs” or other health status data. As used herein, vital signs include but are not limited to respiratory (breathing) rate, the concentration of respired gases, pulse rate, blood pressure, and cardiac electrical activity.

In some embodiments, the method and apparatus may be used to monitor and thus protect the health and lives of infants at risk for the occurrence of sudden infant death syndrome (SIDS). However, those skilled in the art will recognize that method and apparatus are applicable to children, adolescents, adults, the elderly, senior, and animals. For example, adults considered at risk for sleep apnea or adverse cardiovascular events may be monitored using the present method and apparatus. Embodiments may be designed to protect individuals at rest, asleep, or untended. Humans or animals being monitored may be referred to as a “patient” or “subject,” and may be of any age or health status.

The methods an apparatus may also be used to study dream behavior, to monitor a subject's bathroom usage or frequency of changing position in bed, to monitor the amount of time a subject spends in a bed chair, couch, etc, to monitor the frequency and/or severity of convulsions or apneas, to monitor the frequency and/or severity of arrhythmias, or to monitor a bed or other surface for evidence of erotic stimulation.

The methods and apparatus may also be used to determine whether a subject is present in a particular location. In this manner, health-status data may be used to identify a particular subject (e.g., via pattern recognition) to confirm the identity of the subject in the location. The health-status data may also be used only to indicate the presence of any subject in a particular location, e.g., to make sure a baby is in a crib, an elderly patient is in a bed, or a dog is in a kennel, without identifying the subject.

Further embodiments and variation using the present method and apparatus will be apparent to the skilled artisan in view of the disclosure. The methods are apparatus are in no way limited by the description.

### EXAMPLES

1. Apparatus for monitoring heart and respiration rates of a human subject at rest, comprising, in operative condition,

a sensing unit having (i) a fluid or gas-filled pad adapted to be placed on a bed, crib, or chair support, for cushioning at least an upper body portion of a subject lying on or resting against the support, mattress or cushion, and (ii) a pressure sensor in gas or fluid communication with gas or fluid in said unit, for generating electrical signals in response to pressure variations within the gas or fluid in the pad, and

a monitoring unit operatively connected to said pressure sensor, for (i) receiving signals therefrom, (ii) generating from said signals, information about the heart and respiration rates of the subject, and (iii) relaying such information to a monitoring station or individual.

2. The apparatus of example 1, wherein the pad is a fluid-filled pad.

3. The apparatus of example 1, wherein the pad is a gas-filled pad.

4. The apparatus of example 1, which further includes a pressure-control unit comprising a pump in fluid or gas communication with the pad and a controller operatively connected to the pump for maintaining fluid or gas within the pad at a selected pressure or within a defined pressure range.

5. The apparatus of example 1, wherein said pad comprises a single fluid or gas-filled chamber, having a pressure sensor in fluid or gas communication therewith, for generating electrical signals in response to pressure changes within the chamber.

6. The apparatus of example 1, wherein said pad includes at least two independent, fluid or gas-filled chambers, each of which has a pressure sensor in fluid communication therewith, for generating electrical signals in response to pressure changes within the associated chamber.

7. The apparatus of example 1, which further includes an ambient-null device comprising a fluid or gas-filled ambient pad, a weight carried on the ambient pad, for exerting pressure thereon, and an ambient pressure sensor in fluid or gas communication with fluid in the ambient pad, for generating electrical signals in response to pressure changes within the fluid or gas, in response to ambient motion in the vicinity of the subject, wherein said monitoring unit is operatively connected to said ambient pressure sensor, for (i) receiving signals therefrom, and (ii) processing the signals received from the first-mentioned and ambient pressure sensors, to filter such ambient motion from motion related to the subject's heart and respiration rates.

8. The apparatus of 1, wherein said monitoring unit includes a processor operative to (i) generate heart-rate information of the subject, based on time-dependent signals having frequency components in the range from about 0.1 to about 10 Hz, and (ii) generate respiration rate information of the subject based on time-dependent signals having frequency components in the range less than about 1 Hz.

9. The apparatus of example 8, wherein the information generated by the signal processor further includes blood-pressure information.

10. The apparatus of example 9, wherein said pad includes upper and lower independent, fluid or gas-filled chambers, each of which has a pressure sensor in fluid communication therewith, for generating electrical signals in response to pressure changes within the associated chamber, and the information generated by the processor includes information about the orientation of the individual with respect to the pad, based on a characteristic ventral, dorsal or lateral signals produced by processing the two separate signals generated for the two chambers.

11. The apparatus of example 8, wherein said monitoring unit includes a remote monitor, and a transmitter for transmitting such heart and respiration rate information from the processor to the monitor.

12. The apparatus example 1, wherein said pad further includes temperature sensor for measuring the temperature of the individual on the pad.

13. A sensor unit for use with a monitoring unit, for monitoring heart and respiration rates of a human subject at rest, comprising, in operative condition,

a fluid or gas-filled pad adapted to be placed on a bed, crib, or chair support, for cushioning at least an upper-body portion of a subject lying on or resting against the support, and

a pressure sensor in fluid communication with fluid in said unit, for generating electrical signals in response to pressure variations within the fluid or gas, and adapted to be operatively connected to such a monitor.

14. The sensing unit of example 13, wherein said pad includes as single fluid or gas-filled chamber having a pressure sensor in fluid of gas communication therewith, for generating electrical signals in response to pressure changes within the chamber.

15. The sensing unit of example 13, wherein said pad includes at least two independent, fluid-filled chambers, each of which has a pressure sensor in fluid or gas communication therewith, for generating electrical signals in response to pressure changes within the associated chamber.

16. The sensing unit of example 13, wherein for the pad is a fluid-filled pad.

17. The sensing unit of example 13, wherein for the pad is a gas-filled pad.

18. A method for monitoring vital signs, including heart and respiration rates, of a human subject lying on or resting against a bed, crib, or chair support, comprising

placing between the subject and the support, a fluid or gas-filled pad positioned for cushioning at least an upper-body area of the subject,

generating electrical signals in response to pressure variations within the fluid or gas by a pressure sensor in fluid communication with fluid or gas in said pad, and

processing the electrical signals received from the pressure sensor to generate information about the heart and respiration rate of the subject.

Apparatus for remotely monitoring heart and respiration rates of a human subject lying on or resting against a bed, crib, or chair support, comprising

a pad adapted to the placed between the subject and the support, for cushioning at least an upper body portion of the individual,

a sensor on said pad for generating motion-related signals caused by the subject's heartbeat and breathing,

a processor operatively connected to said sensor, for (i) receiving time-dependent signals therefrom, and (ii) generating heart-rate information of the subject, based on received time-dependent signals in the range from about 0.1 to about 10 Hz, and respiration rate information of the subject, based on received timed-dependent signals in the range less than about 1 Hz,

a remote monitor for use by an individual in monitoring said subject, and

a transmitter for transmitting such subject information from the processor to the individual.

19. The apparatus of example 18, which further includes an ambient-motion device for generating signals related to ambient motion in the vicinity of the subject, and said processor is operatively connected to said device, for processing the signals received from the device, to filter such ambient motion from motion related to the subject's heart and respiration rates.

20. Apparatus for monitoring vital signs, including heart and respiration rates, of a human subject lying on or resting against a bed, crib, or chair support, comprising

a pad adapted to the placed between the subject and the support, for cushioning at least an upper body portion of the individual, said pad comprising

a pair of confronting plates, one adapted to be supported on the mattress, and the other adapted for contact with the chest area of the individual, said plates being spaced apart for relative lateral movement in an XY plane and relative vertical movement in the Z direction,

(iii) connecting the two plates, an L-shaped connector attached at opposite ends to the opposing plates and having a pair of laterally extending, orthogonally disposed arms, a strain gauge carried on each arm, in an XY plane, and a strain gauge carried on one of the arms, in a vertical plane, and

(b) a monitoring unit operative to transmit to a remote user, information about the heart rate of the individual, based on signals received from the pad's lateral-movement strain gauge devices, and about the respiration rate of the individual, based on signals received from the pad's vertical-movement strain gauge(s).

21. The apparatus of example 20, wherein said two opposing plates are substantially rectangular, and connected by said L-shaped connectors in the region of each of the four corners of the two plates.

22. The apparatus of example 21, which further includes a vertical-movement strain gauge connecting the two plates, for generating information about the weight applied by the individual on the pad.

23. The apparatus of example 21, wherein said monitoring unit includes a processor operative to (i) wherein said monitoring unit includes a signal processor operative to (i) generate heart-rate information of the subject, based on time-dependent signals received from each of the plural lateral-movement strain-gauge devices, and having frequency components in the range from about 0.1-10 Hz, and (ii) generate respiration rate information of the subject based on timed-dependent signals having frequency received from the at least one of the vertical-movement strain gauge(s), and having frequency components in the range less than about 1 Hz.

24. The apparatus of example 23, wherein said monitoring unit includes a remote monitor, and a transmitter for transmitting such heart rate and respiration rate information from the processor to the monitor.

25. The apparatus example 24, wherein said pad further includes temperature sensor for measuring the temperature of the individual on the pad.

26. The apparatus of example 24, which further includes a weighted strain gauge adapted for attachment to the bed or crib, independent of said pad, for detecting movement of the bed or crib, independent of movement within the pad, and the monitoring unit is operative to remove such independent movement from pad movement detected by the pad strain gauges.

27. Apparatus for determining the presence of a subject, comprising:

a sensing unit having (i) a fluid or gas-filled pad adapted to be placed on a bed, crib, or chair support, for cushioning at least an upper body portion of a subject lying on or resting against the support, mattress or cushion, and (ii) a pressure sensor in fluid communication with fluid in said unit, for generating electrical signals in response to pressure variations within the fluid in the pad, and

a monitoring unit operatively connected to said pressure sensor, for (i) receiving signals therefrom, (ii) generating from said signals, information about the presence of the subject and (iii) relaying such information to a monitoring station or individual.

28. A sensor unit for use with a monitoring unit, for detecting the presence of a subject, comprising:

a fluid or gas-filled pad adapted to be placed on a bed, crib, or chair support, for cushioning at least an upper-body portion of a subject lying on or resting against the support, and

a pressure sensor in fluid communication with fluid in said unit, for generating electrical signals in response to pressure variations within the fluid or gas, and adapted to be operatively connected to such a monitor.

29. A method for detecting the presence of a subject on or in a bed, crib, or chair support, comprising:

placing on or in the bed, crib, or chair support a fluid or gas-filled pad positioned for cushioning at least an upper-body area of the subject,

generating electrical signals in response to pressure variations within the fluid or gas by a pressure sensor in fluid communication with fluid or gas in said pad, and

processing the electrical signals received from the pressure sensor to generate information about the presence of the subject.

30. Apparatus for monitoring the presence of a subject lying on or resting against a bed, crib, or chair support, comprising:

a pad adapted to the placed between the subject and the support, for cushioning at least an upper body portion of the individual, said pad comprising

a pair of confronting plates, one adapted to be supported on the mattress, and the other adapted for contact with the chest area of the individual, said plates being spaced apart for relative lateral movement in an XY plane and relative vertical movement in the Z direction,

(iii) connecting the two plates, an L-shaped connector attached at opposite ends to the opposing plates and having a pair of laterally extending, orthogonally disposed arms, a strain gauge carried on each arm, in an XY plane, and a strain gauge carried on one of the arms, in a vertical plane, and

(b) a monitoring unit operative to transmit to a remote user, information about the presence of the subject, based on signals received from the pad's strain gauge devices.

1. (canceled)

2. A sensor apparatus comprising:
an air bladder configured to be positioned under a mattress, wherein the air bladder has a bladder top and a bladder bottom that combine to define an air chamber of the air bladder, wherein the air chamber of the air bladder has a substantially rectangular outer perimeter portion when viewed from above or below the air bladder, wherein the outer perimeter portion has first, second, third, and fourth edges, wherein the air bladder defines a bladder inlet to the air chamber that is positioned along the first edge of the outer perimeter of the air chamber of the air bladder, wherein the bladder top is connected to the bladder bottom so as to define a plurality of channels that extend longitudinally in a direction that is parallel to the second and fourth edges and that is perpendicular to the first and third edges, wherein the air bladder is configured to allow air flow between the bladder inlet and the channels, wherein the air bladder is sized smaller than a surface area of a mattress and is sized large enough to sense a user's heart and lungs when positioned under a mattress, wherein the air bladder is made of an inflatable and substantially air-tight plastic material;
a housing made of a rigid plastic material;
an electrical air pump positioned in the housing, in fluid communication with the bladder inlet of the air bladder, and operable to control fluid pressure within the air bladder;
an air pressure sensor positioned in the housing, in fluid communication with the bladder inlet of the air bladder, and configured to sense fluid pressure of the air bladder and to generate a sensor signal as a function of sensed fluid pressure of the air bladder;
a processing unit in electrical communication with the air pressure sensor and configured to:
receive the sensor signal from the air pressure sensor; and
convert the received sensor signal to data;
a transmitter in data communication with the processing unit configured to transmit the data; and
a cover configured to cover the air bladder for comfort without substantially insulating the sensor apparatus from sensing vibrations generated by heart and lung function.

3. The sensor apparatus of claim 2, wherein the air pressure sensor is configured to sense incident fluid pressure waves caused by cardiopulmonary activity of a user lying on the mattress.

4. The sensor apparatus of claim 2, wherein the transmitter is configured to transmit the data to a server computer configured to receive the transmitted data, store the received data, and provide status signals indicating parameters of a user lying on the mattress, wherein the transmitter is a wired transmitter that is integral with the air bladder and is in wired communication with the processing unit.

5. The sensor apparatus of claim 4, wherein the parameters comprise a user's heart rate, respiratory rate, the user's time in bed, amount of time spent in REM sleep, and a sleep quotient based in part on the user's time in bed.

6. The sensor apparatus of claim 4, wherein the server computer is further configured to calculate a sleep quotient based on one or more of parameters, wherein the sleep quotient is a score indicative of quality of a night's sleep by the user.

7. The sensor apparatus of claim 2, further comprising an audio sensor configured for detecting snoring.

8. The sensor apparatus of claim 2, wherein the electrical air pump and the air pressure sensor are in fluid communication with the air bladder along a common flow path.

9. A bed system comprising the sensor apparatus of claim 2, a mattress, a bed frame, wherein the mattress is positioned on the bed frame, wherein the sensor apparatus is positioned between the mattress and the bed frame with the air bladder positioned entirely under the mattress at a location that is longitudinally between a head of the mattress and a lateral midline of the mattress such that the air bladder is positioned at a location under which a user's heart and lungs would be expected.

10. The sensor apparatus of claim 2, wherein the transmitter is a wireless transceiver configured to transmit the data to a server computer configured to receive the transmitted data, store the received data, and provide status signals indicating parameters of a user lying on the mattress.

11. The sensor apparatus of claim 10, wherein the parameters comprise a user's heart rate, respiratory rate, the user's time in bed, amount of time spent in REM sleep, and a sleep quotient based in part on the user's time in bed.

12. The sensor apparatus of claim 11, wherein the server computer is further configured to calculate a sleep quotient based on one or more of parameters, wherein the sleep quotient is a score indicative of quality of a night's sleep by the user.

13. A sleep sensor system comprising:
an air bladder configured to be positioned under a mattress, wherein the air bladder has a bladder top and a bladder bottom that combine to define an air chamber of the air bladder, wherein the air chamber of the air bladder has a substantially rectangular outer perimeter portion when viewed from above or below the air bladder, wherein the outer perimeter portion has first, second, third, and fourth edges, wherein the air bladder defines a bladder inlet to the air chamber that is positioned along the first edge of the outer perimeter of the air chamber of the air bladder, wherein the bladder top is connected to the bladder bottom so as to define a plurality of channels that extend longitudinally in a direction that is parallel to the second and fourth edges and that is perpendicular to the first and third edges, wherein the air bladder is configured to allow air flow between the bladder inlet and the channels, wherein the air bladder is sized smaller than a surface area of a mattress and is sized large enough to sense a user's heart and lungs when positioned under a mattress, wherein the air bladder is made of an inflatable and substantially air-tight plastic material;
a cover configured to cover the air bladder for comfort without substantially insulating the sleep sensor system from sensing vibrations generated by heart and lung function for comfort;
a housing made of a rigid plastic material;
an electrical air pump positioned in the housing, in fluid communication with the air bladder, and operable to control a fluid pressure within the air bladder;
an air pressure sensor positioned in the housing, in fluid communication with the bladder inlet of the air bladder, and configured to sense fluid pressure of the air bladder and to generate a pressure sensor signal as a function of sensed fluid pressure of the air bladder, wherein the air pressure sensor is configured to sense incident fluid pressure waves caused by cardiopulmonary activity of a user lying on the mattress;
a temperature sensor;
a light sensor;
an audio sensor;
an alarm system;
a processing unit, comprising a processor, in electrical communication with the air pressure sensor, and configured to:
receive the pressure sensor signal from the air pressure sensor;
receive a temperature sensor signal from the temperature sensor;
receive a light sensor signal from the light sensor;
receive an audio sensor signal from the audio sensor;
convert the received sensor signals to data;
determine, based on the data, presence of the user on the mattress;
determine, based on the data, absence of the user on the mattress;
determine, based on the data, a length of sleep by the user;
determine, based on the data, the user's heart beats;
determine, based on the data, the user's breaths;
a wireless transmitter in data communication with the processing unit and configured to transmit the data using at least one of an IEEE 802.11 protocol and a BLUETOOTH protocol.

14. The sleep sensor system of claim 13, further comprising:
a server computer system comprising one or more processors and a computer-readable memory storing a monitoring software program, the monitoring software program comprising instructions that instruct the one or more processors to:
receive the data;
determine a user heart rate of the user based on the data;
determine a user respiration rate of the user based on the data;
determine a total time in bed of the user based on the data;
determine an amount of time spent in rapid eye movement (REM) sleep based on the data;
determine a luminosity based on the data;
determine a temperature based on the data;
determine an audio level based on the data;
calculate a sleep quotient based on all of the determined user heart rate, the determined user respiration rate, and the determined total time in bed, wherein the sleep quotient is a score indicative of quality of a night's sleep by the user; and
transmit a total time in bed signal based on the user's determined total time in bed and is configured such that the total time is displayable on a display screen;
transmit a heart rate signal based on the user's determined heart rate and is configured such that the heart rate is displayable on a display screen;
transmit a respiratory rate signal based on the user's determined respiration rate and is configured such that the respiration rate is displayable on a display screen;
transmit a REM sleep time signal based on the user's determined amount of time spent in REM sleep and is configured such that the REM sleep time is displayable on a display screen; and
transmit a sleep quotient signal that is based on the user's determined sleep quotient and is configured such that the sleep quotient is displayable on a display screen.

15. The sleep sensor system of claim 13, wherein the processing unit further comprises an alarm clock.

16. A sensor apparatus comprising:
an air bladder configured to be positioned under a mattress and above a bed frame, wherein the air bladder has a bladder top and a bladder bottom that combine to define an air chamber of the air bladder, wherein the chamber of the air bladder has a substantially rectangular outer perimeter portion when viewed from above or below the air bladder, wherein the outer perimeter portion has first, second, third, and fourth edges, wherein the air bladder defines a bladder inlet to the air chamber that is positioned along the first edge of the outer perimeter of the air chamber of the air bladder, wherein the bladder top is connected to the bladder bottom so as to define a plurality of channels that extend longitudinally in a direction that is parallel to the second and fourth edges and that is perpendicular to the first and third edges, wherein the air bladder is configured to allow air flow between the bladder inlet and the channels, wherein the air bladder is sized smaller than a surface area of a mattress and is sized large enough to sense a user's heart and lungs when positioned under a mattress, wherein the air bladder is made of an inflatable and substantially air-tight plastic material;
a pressure control unit comprising:
a housing made of a rigid plastic material;
an electrical air pump positioned in the housing, in fluid communication with the bladder inlet of the air bladder, and operable to control fluid pressure within the air bladder;
an air pressure sensor positioned in the housing, in fluid communication with the bladder inlet of the air bladder and configured to sense fluid pressure of the air bladder and to generate a sensor signal as a function of sensed fluid pressure of the air bladder, wherein the air pressure sensor is configured to sense incident fluid pressure waves caused by cardiopulmonary activity of a the user lying on a top of the mattress;
a processing unit integral with the air bladder and in electrical communication with the air pressure sensor and the electrical air pump and configured to:
receive the sensor signal from the air pressure sensor;
operate the electrical air pump to maintain a predetermined pressure within the air bladder; and
convert the received sensor signal to data;
a wireless transmitter in data communication with the processing unit, integral with the air bladder, and configured to transmit the data using at least one of an IEEE 802.11 protocol and a BLUETOOTH protocol; and
a cover configured to cover the air bladder for comfort without substantially insulating the sensor apparatus from sensing vibrations generated by heart and lung function of the user lying on the top of the mattress.

17. The sensor apparatus of claim 16, wherein the wireless transmitter transmits the data to a server computer configured to receive the transmitted data, store the received data, and provide status signals indicating parameters of a user lying on the mattress.

18. A method of sensing one or more sleep parameters, the method comprising:
placing an air bladder under a mattress, wherein the air bladder has a bladder top and a bladder bottom that combine to define an air chamber of the air bladder, wherein the air chamber of the air bladder has a substantially rectangular outer perimeter portion when viewed from above or below the air bladder, wherein the outer perimeter portion has first, second, third, and fourth edges, wherein the air bladder defines a bladder inlet to the air chamber that is positioned along the first edge of the outer perimeter of the air chamber of the air bladder, wherein the bladder top is connected to the bladder bottom so as to define a plurality of channels that extend longitudinally in a direction that is parallel to the second and fourth edges and that is perpendicular to the first and third edges, wherein the air bladder is configured to allow air flow between the bladder inlet and the channels, wherein the air bladder is sized smaller than a surface area of a mattress and is sized large enough to sense a user's heart and lungs when positioned under a mattress, wherein the air bladder is made of an inflatable and substantially air-tight plastic material;
controlling, by an electrical air pump in fluid communication with the bladder inlet of the air bladder, operable to control fluid pressure within the air bladder, and housed within a housing made of a rigid plastic material, a fluid pressure within the air bladder;
sensing, by an air pressure sensor positioned in the housing, in fluid communication with the air bladder and configured to sense fluid pressure of the air bladder and to generate a sensor signal as a function of sensed fluid pressure of the air bladder, fluid pressure of the air bladder, wherein sensing the fluid pressure within the air bladder comprises sensing incident fluid pressure waves caused by cardiopulmonary activity of a user lying on the mattress and wherein the air bladder is covered by a cover configured to cover the air bladder for comfort without substantially insulating the pressure sensor from sensing vibrations generated by heart and lung function of a user on a top of the mattress;
generating, by the air pressure sensor, a sensor signal as a function of sensed fluid pressure of the air bladder;
receiving, by a processing unit integral with the air bladder and in electrical communication with the air pressure sensor, the sensor signal from the air pressure sensor;
converting, by the processing unit, the received sensor signal to data indicative of the user's heart rate, the user's respiratory rate, and the user's time in bed; and
transmitting, by a wireless transmitter in data communication with the processing unit and integral with the air bladder, the data.

19. The method of claim 18, further comprising:
receiving, by a server computer, the transmitted data;
storing, by the server computer, the received data; and
providing, by the server computer, status signals indicating parameters of the user lying on the mattress.

20. The method of claim 19, wherein the parameters comprise the user's heart rate, the user's respiratory rate, the user's time in bed, amount of time spent in REM sleep, and a sleep quotient based in part on the user's time in bed.

21. The method of claim 20, further comprising:
receiving, by a user device, the status signals; and
presenting, at a user interface of the user device, visual or audible representations of the sleep quotient, the heart rate, the respiration rate, and the amount of time spent in REM sleep.

22. The method of claim 18, wherein the air bladder is portable and configured to be rolled, the method further comprising:
unfolding the air bladder before placing it under the mattress; and
placing a processing unit on a night stand, wherein the processing unit is configured to communicate with the air pressure sensor.

23. The method of claim 18, wherein transmitting comprises transmitting, by the transmitter, the data to a server computer configured to receive the transmitted data, store the received data, and provide status signals indicating parameters of a user lying on the mattress, wherein the transmitter is a wired transmitter that is integral with the air bladder and is in wired communication with the processing unit.

24. The method of claim 23, wherein the parameters comprise a user's heart rate, respiratory rate, and the user's time in bed, the method further comprising determining a sleep quotient based on the parameters, wherein the sleep quotient is a score indicative of quality of a night's sleep by the user.

25. The method of claim 18, wherein the transmitter is integral with the air bladder and is a wireless transceiver configured to transmit the data to a server computer configured to receive the transmitted data, store the received data, and provide status signals indicating parameters of a user lying on the mattress, wherein the parameters comprise the user's heart rate, respiratory rate, the user's time in bed, amount of time spent in REM sleep, and a sleep quotient based in part on the user's time in bed.
